(19) United States
(12) Patent Application Publication
(10) Pub. No.: US 2023/0338493 A1
(43) Pub. Date: Oct. 26, 2023

(54) DECTIN-1 (CLEC7A) SINGLE NUCLEOTIDE POLYMORPHISM AS A BIOMARKER FOR PREDICTING ANTIBODY RESPONSE WHEN USING BETA-GLUCAN AS A VACCINE ADJUVANT

(71) Applicant: MEMORIAL SLOAN KETTERING CANCER CENTER, New York, NY (US)

(72) Inventors: Irene Y. Cheung, New York, NY (US); Nai-Kong V. Cheung, New York, NY (US)

(21) Appl. No.: 18/001,246

(22) PCT Filed: Jun. 9, 2021
(86) PCT No.: PCT/US2021/036544

§ 371 Date: Dec. 8, 2022

Related U.S. Application Data
(60) Provisional application No. 63/037,818, filed on Jun. 11, 2020.

(51) Int. Cl. A61K 39/00; C12Q 1/6886; A61K 39/39; A61P 35/00

(52) U.S. Cl. CPC A61K 39/001171; C12Q 1/6886; A61K 39/39; A61P 35/00; C12Q 2600/106; C12Q 2600/156; A61K 2039/6081; A61K 2039/55577; A61K 2039/575; A61K 2039/55583; A61K 2039/542; A61K 2039/70

(57) ABSTRACT

The present disclosure relates generally to methods for determining whether a patient will show an enhanced immunogenic response to vaccines when using β-glucan as a vaccine adjuvant. Kits for use in practicing the methods are also provided

### CROSS-REFERENCE TO RELATED APPLICATIONS

This application is the U.S. National Stage Application of PCT/US2021/036544, filed Jun. 9, 2021, which claims the benefit of and priority to U.S. Provisional Patent Application No. 63/037,818, filed Jun. 11, 2020, the contents of which are incorporated by reference herein in its entirety.

### STATEMENT OF GOVERNMENT SUPPORT

This invention was made with government support under CA008748 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jul. 15, 2021, is named 115872-2233_SL.txt and is 2,488 bytes in size.

### TECHNICAL FIELD

The present technology relates to methods for predicting whether a patient will show an enhanced immunogenic response to vaccines when using β-glucan as a vaccine adjuvant. These methods are based on detecting the presence of a wild-type rs3901533 SNP in at least one CLEC7A polynucleotide in a patient. Kits for use in practicing the methods are also provided.

### BACKGROUND

The following description of the background of the present technology is provided simply as an aid in understanding the present technology and is not admitted to describe or constitute prior art to the present technology.

Besides cancer, pandemics and biologic warfare are global threats. Cancer vaccines and infectious disease vaccines (from viruses, fungi and bacteria) can be developed rationally and expeditiously, but their efficacy depends on the human host. Ability to mount an effective immune response in a sizable percentage of human subjects is key to the success of such vaccines. Yet, even with the right antigen formulation, human immune response is often unpredictable and typically insufficient for these vaccines to be protective. Examples include influenza, Ebola, tuberculosis, HIV, malaria, corona virus and most cancer vaccines. One major hurdle is the lack of an effective and safe vaccine adjuvant to boost the immune responses (O'Hagan D T et al., Curr Opin Immunol 47:93-102 (2017)). Adjuvants currently in use or in development often rely on empiric observations, without a clear understanding of the cellular or molecular mechanism of action. It is increasingly evident that most effective adjuvants activate T and B cell responses by engaging cells in the innate immune system (Coffman et al., Immunity 33:492-503 (2010)), instead of direct effects on the lymphocytes. Besides infectious diseases, adjuvants for cancer (Saxena & Bhardwaj, Curr Opin Immunol 47:35-43 (2017)) and vaccines for Alzheimer's disease (Novak et al., Lancet Neurol 16:123-134 (2017)) are also suboptimal. Accordingly, there is an urgent need for reliable biomarkers that predict whether an adjuvant can be safely and effectively deployed in immune disadvantaged populations such as children, the elderly, and the immunocompromised. See e.g., Kollmann & Marchant, Trends Immunol 37:523-534 (2016); Mohr & Siegrist, Curr Opin Immunol 41:1-8 (2016); Schaffner et al., Am J Med 131(8):865-873 (2018)).

### SUMMARY OF THE PRESENT TECHNOLOGY

In one aspect, the present disclosure provides a method for identifying a subject that will show an enhanced immunogenic response to a vaccine comprising: detecting the presence of a wild-type rs3901533 SNP (e.g., GRCh38.p12 chr12:10124484A) in at least one CLEC7A polynucleotide in a biological sample obtained from the subject, wherein the presence of the wild-type rs3901533 SNP in at least one CLEC7A polynucleotide indicates that the subject will show an enhanced immunogenic response to a vaccine. In certain embodiments, the method further comprises administering to the subject an effective amount of a vaccine and an effective amount of a yeast beta-glucan comprising a plurality of β-(1,3) side chains linked to a β-(1,3) backbone via β-(1,6) linkages, and wherein the yeast beta-glucan has a range of average molecular weights from about 6 kDa to about 30 kDa; optionally wherein the vaccine comprises at least one antigen that is linked to a carrier, and optionally wherein the antigen is a peptide, a polypeptide, a nucleic acid, a carbohydrate, a lipid, or a whole tumor cell. In some embodiments, the SNP is detected via next-generation sequencing, PCR, real-time quantitative PCR (qPCR), digital PCR (dPCR), Southern blotting, Reverse transcriptase-PCR (RT-PCR), Northern blotting, microarray, dot or slot blots, in situ hybridization, or fluorescent in situ hybridization (FISH). The biological sample may comprise genomic DNA and/or peripheral blood mononuclear cells. Additionally or alternatively, in some embodiments, the immunogenicity of the vaccine in the subject is increased compared to that observed in a control subject that does not harbor the wild-type rs3901533 SNP.

Additionally or alternatively, in some embodiments, the vaccine is a poorly immunogenic antigen-specific vaccine or a whole cell tumor vaccine. In any and all embodiments of the methods disclosed herein, the at least one antigen is associated with a disease or infection. Examples of such diseases and infections include, but are not limited to neurodegenerative disease, Alzheimer's Disease, melanoma, neuroblastoma, glioma, small cell lung cancer, t-ALL, breast cancer, brain tumors, retinoblastoma, Ewing's sarcoma, osteosarcoma, ovarian cancer, non-Hodgkin's lymphoma, Epstein-Barr related lymphoma, Hodgkin's lymphoma, leukemia, epidermoid carcinoma, prostate cancer, renal cell carcinoma, transitional cell carcinoma, lung cancer, colon cancer, liver cancer, stomach cancer, gastrointestinal cancer, pancreatic cancer, HIV, tuberculosis, malaria, influenza, Ebola, chicken pox, Hepatitis B, HPV, tetanus, pneumococcus, measles, mumps, rubella, influenza, polio, diphtheria, tetanus, pertussis, Rous Sarcoma Virus, rabies, and rotavirus.

Additionally or alternatively, in some embodiments, the structure of the at least one antigen is

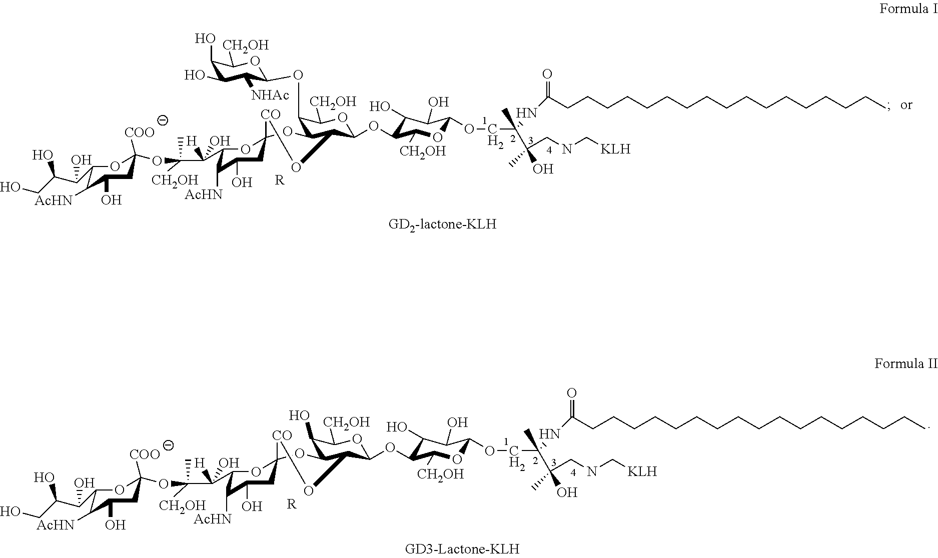

Additionally or alternatively, in some embodiments, the at least one antigen is inactivated, partially purified or recombinant hemagglutinin (HA) protein or fucosyl GM1. Examples of the carrier include keyhole limpet hemocyanin, serum globulins, serum albumins, and ovalbumins.

Additionally or alternatively, in some embodiments, the vaccine and the yeast beta-glucan are administered separately, simultaneously or sequentially. In certain embodiments, the vaccine is administered intravenously, intramuscularly, intraarterially, intrathecally, intracapsularly, intraorbitally, intradermally, intraperitoneally, transtracheally, subcutaneously, intracerebroventricularly, orally or intranasally. In some embodiments, the yeast beta-glucan is administered intravenously, intramuscularly, intraarterially, intrathecally, intracapsularly, intraorbitally, intradermally, intraperitoneally, transtracheally, subcutaneously, intracerebroventricularly, orally or intranasally.

Additionally or alternatively, in some embodiments, administration of the vaccine and the yeast beta-glucan results in about a 10-fold increase in therapeutic antibody titer levels in the subject compared to that observed in the subject prior to administration of the vaccine and the yeast beta-glucan. In certain embodiments, administration of the vaccine and the yeast beta-glucan results in the persistence of therapeutic antibody titer levels in the subject. In any of the above embodiments of the methods disclosed herein, administration of the yeast beta-glucan prolongs survival and/or prevents tumor recurrence in the subject.

In another aspect, the present disclosure provides a method for treating a metastasis-prone cancer, a neurodegenerative disease, or an infection in a subject in need thereof comprising administering to the subject an effective amount of a vaccine and an effective amount of a yeast beta-glucan comprising a plurality of β-(1,3) side chains linked to a β-(1,3) backbone via β-(1,6) linkages, wherein the yeast beta-glucan has a range of average molecular weights from about 6 kDa to about 30 kDa, and wherein the subject harbors a wild-type rs3901533 (e.g., GRCh38.p12 chr12:10124484A) SNP in at least one CLEC7A polynucleotide. In some embodiments, the vaccine comprises at least one antigen that is optionally linked to a carrier, and wherein the antigen is a peptide, a polypeptide, a nucleic acid, a carbohydrate, a lipid, or a whole tumor cell. The SNP may be detected via next-generation sequencing, PCR, real-time quantitative PCR (qPCR), digital PCR (dPCR), Southern blotting, Reverse transcriptase-PCR (RT-PCR), Northern blotting, microarray, dot or slot blots, in situ hybridization, or fluorescent in situ hybridization (FISH). Additionally or alternatively, in some embodiments, administration of the vaccine and the yeast beta-glucan protects the subject from metastasis, elevates helper T cell response to vaccines, and/or promotes progression free survival in the subject.

Examples of metastasis-prone cancers, neurodegenerative diseases, or infections include, but are not limited to Alzheimer's Disease, melanoma, neuroblastoma, glioma, small cell lung cancer, t-ALL, breast cancer, brain tumors, retinoblastoma, Ewing's sarcoma, osteosarcoma, ovarian cancer, non-Hodgkin's lymphoma, Epstein-Barr related lymphoma, Hodgkin's lymphoma, leukemia, epidermoid carcinoma, prostate cancer, renal cell carcinoma, transitional cell carcinoma, lung cancer, colon cancer, liver cancer, stomach cancer, gastrointestinal cancer, pancreatic cancer, HIV, tuberculosis, malaria, influenza, Ebola, chicken pox, Hepatitis B, HPV, tetanus, pneumococcus, measles, mumps, rubella, influenza, polio, diphtheria, tetanus, pertussis, Rous Sarcoma Virus, rabies, and rotavirus. Additionally or alternatively, in some embodiments, wherein the yeast beta-glucan is administered intravenously, intramuscularly, intraarterially, intrathecally, intracapsularly, intraorbitally, intradermally, intraperitoneally, transtracheally, subcutaneously, intracerebroventricularly, orally or intranasally.

In any of the preceding embodiments of the methods disclosed herein, the subject is an immunocompromised subject, a pediatric subject, a geriatric subject, a relapsed subject, or a healthy subject. In certain embodiments, the subject has been exposed to chemotherapy or radiotherapy. Additionally or alternatively, in some embodiments, the subject is human. In any and all embodiments of the methods disclosed herein, the subject is homozygous (A/A) or heterozygous (C/A) for the wild-type rs3901533 SNP. In any and all embodiments of the methods disclosed herein, the rs3901533 SNP is detected using one or more detectably labelled probes comprising a nucleic acid sequence of SEQ ID NO: 1, SEQ ID NO: 2, SEQ ID NO: 3, or SEQ ID NO: 4.

### BRIEF DESCRIPTION OF THE DRAWINGS

FIG. 1A shows that Dectin-1 contains a single carbohydrate recognition domain, a stalk region, a transmembrane domain and a cytoplasmic tail that contains an ITAM-like motif. Dectin-1 has two major isoforms in mice and humans, isoform A and isoform B, and six minor isoforms (not shown). Isoform A and isoform B of Dectin-1 differ with respect to the presence of the stalk region. FIG. 1B shows that upon recognition of β-glucan on fungi, Dectin-1 mediates Syk-dependent NFAT and NF-κB pathways, and the Syk-independent Raf-1 pathway to promote the production of cytokines and chemokines, which in turn promote antifungal defense through Th1 and Th17 responses. In addition, Dectin-1 signaling also promotes early release of arachidonic acid and eicosanoid production. Dectin-1 also mediates antifungal responses through phagocytosis, reactive oxygen species (ROS) production and inflammasome activation, which are essential in the cleavage and activation of pro-IL-1β to IL-1β.

FIG. 2 shows an exemplar molecular structure of the yeast beta-glucans described herein (n is an integer from 0 to about 50, m is an integer from about 35 to about 2000).

FIG. 3 shows a schematic of the vaccine protocol employed in the Examples described herein.

FIG. 4 shows the probability of progression free survival (PFS) and overall survival (OS) upon achieving clinical remission (CR2) among 101 high risk neuroblastoma patients with prior disease progression who responded to the vaccine.

FIGS. 5A-5B show the anti-GD2 IgG1 titers during vaccine treatment in neuroblastoma patients who received oral β-glucan compared with vaccine treated melanoma patients who had not received β-glucan.

FIG. 6A shows the anti-GD3 associated IgG1 antibody titers in patients on vaccine/β-glucan treatment (mean±s.e.m.). FIG. 6B shows the anti-GD2 associated IgG1 antibody titers in patients on vaccine/β-glucan treatment (mean±s.e.m.). FIG. 6C shows the anti-GD2 associated IgM antibody titers in patients on vaccine/β-glucan treatment (mean±s.e.m.).

FIG. 7 shows persistence of antibody titers among patients that had completed vaccine treatment and subsequently went off β-glucan.

FIG. 8A shows that induction of high anti-GD2-IgG1 titers by week 8 was correlated with improved PFS among 101 patients with prior disease progressions (CR2). FIG. 8B shows that induction of high anti-GD2-IgG1 titers by week 8 was correlated with improved OS among 101 patients with prior disease progressions (CR2).

FIG. 9A shows the correlation of anti-GD2-IgG1 titers with PFS in patients that relapsed once. FIG. 9B shows the correlation of anti-GD2-IgG1 titers with PFS in patients that exhibited multiple relapses. These data demonstrate that anti-GD2 IgG1 can serve as an early outcome predictor for patients with only one prior relapse (N=66) vs multiple relapses before vaccine (N=36).

FIG. 10A shows the correlation of anti-GD2-IgG1 titers with OS in patients that relapsed once. FIG. 10B shows the correlation of anti-GD2-IgG1 titers with OS in patients that exhibited multiple relapses. These data demonstrate that anti-GD2 IgG1 can serve as an early outcome predictor for patients with only one prior relapse (N=66) vs multiple relapses before vaccine (N=36).

FIG. 11A shows the lack of correlation between anti-GD2-IgG1 antibody response titers and anti-GD3-IgG1 antibody response titers. FIG. 11B shows the lack of correlation between anti-GD2-IgG1 antibody response titers and anti-GD2-IgM antibody response titers. FIG. 11C shows the lack of correlation between anti-GD2-IgG1 antibody response titers and anti-KLH-IgG1 antibody response titers.

FIG. 12A shows the lack of correlation between high anti-GD3 IgG1 titer antibody response titers and PFS. FIG. 12B shows the lack of correlation between high anti-GD3 IgG1 titer antibody response titers and OS.

FIG. 13 shows the probability of PFS and OS among 80 high risk neuroblastoma patients without prior disease progression (CR1) and treated with vaccine.

FIG. 14 shows a comparison of anti-GD2 associated IgG1 titer among patients who received β-glucan at week 6 (late, group 1=control arm) versus those who received β-glucan upfront (early, group 2).

FIG. 15 shows PFS of neuroblastoma patients in a randomized study, group 1=controlled arm, with late glucan and group 2=early glucan.

FIG. 16 shows the generic structure of a yeast beta-glucan comprising a plurality of β-(1,3) side chains linked to a β-(1,3) backbone via β-(1,6) linkages. R₁, R₂ and R₃ are independently H or R (formula also shown in FIG. 16), n is an integer from 0 to about 50, m is an integer from about 35 to about 2000, each of the m glucose units may have different R₂ and n, and there is at least one R group on the glucan.

FIG. 17 shows the 1H NMR spectrum of a typical yeast soluble beta-glucan (SBG) sample (Biotec Pharmacon ASA, Tromso, Norway). An SBG sample was dissolved in DMSO-d6 at a concentration of approximately 20 mg/ml and with a few drops of TFA-d added. The spectrum (cut-out from 2.7 to 5.5 ppm) was collected over 2 hours on a JEOL ECX 400 NMR spectrometer at 80° C. Chemical shifts were referenced to residual proton resonance from the DMSO-d6 at 2.5 ppm, and the spectrum was baseline corrected.

FIG. 18 shows the viscosity profile of SBG. Profiles for a 2% solution of SBG at 20° C. or 30° C. at different shear rates are shown. Glycerol (87% solution) was used as a reference solution.

FIG. 19 shows the characteristics of the one hundred and two high risk neuroblastoma (HR-NB) patients with prior disease progressions (Clinicaltrials.gov NCT00911560) described in the Examples herein.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al., eds. (2007) Current Protocols in Molecular Biology; the series Methods in Enzymology (Academic Press, Inc., N.Y.); MacPherson et al., (1991) PCR 1: A Practical Approach (IRL Press at Oxford University Press); MacPherson et al., (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al., eds (1996) Weir's Handbook of Experimental Immunology.

Successful antibody responses to vaccines should be long-lived, requiring infrequent or no additional boosting to sustain host protection. However, some antigens such as carbohydrates are unable to achieve adequate seroconversion rates or protective antibody titers. For example, GD2 and GD3 are self-antigens present on normal human tissues, although their expression is significantly higher in neuroectodermal tumors, including neuroblastoma, osteosarcoma, soft tissue sarcoma, small cell lung cancer, retinoblastoma and brain tumors. As self-antigens, GD2 and GD3 are not immunogenic in humans. To build a GD2 and GD3-specific vaccine for neuroblastoma, GD2-lactone-KLH and GD3-lactone-KLH were constructed. These lactone forms can induce antibodies to react with native GD2 and GD3 polypeptides. In large studies where these GD2-L-KLH/GD3-L-KLH vaccine (abbreviated as GD2/GD3 bivalent vaccine) is combined with QS21 (adjuvant) as subcutaneous injections (6 injections over 7 months), very low titers, primarily of the IgM class, of anti-GD2 or anti-GD3 antibody responses were observed. These previous randomized studies did not show vaccine benefit on patient survival (Eggermont et al., Journal of Clinical Oncology 31:3831-3837 (2013); Carvajal et al., Journal of Clinical Oncology 32:10520-10520 (2014); Danishefsky et al., Accounts of Chemical Research 48:643-652 (2015); Huang C-S et al., Journal of Clinical Oncology 34:1003-1003 (2016)).

The present disclosure explores the efficacy of the GD2/GD3 bivalent vaccine in pediatric patients with a history of receiving chemotherapy, highlighting the immunocompromised state in a young child with immature lymphoid systems, both characteristics challenging for any vaccine or adjuvant. The present disclosure demonstrates that β-glucan can greatly enhance the T cell dependent IgG antibody response to a vaccine (e.g., GD2/GD3 bivalent vaccine), through the dectin-1 receptor mechanism, and that the rs3901533 SNP serves as an accurate and reliable biomarker for antibody response to a vaccine when β-glucan is used as an adjuvant. These rs3901533 SNP-seroconversion results are significant and unexpected given that Table 3 the present disclosure further demonstrates that not all dectin-1 SNPs (e.g., rs16910526 (chr12:10118488 (GRCh38.p12)), rs7309123 (chr12:10119994 (GRCh38.p12))) are correlated with seroconversion.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

The positions of each of dectin-1 SNPs disclosed herein are based on the consensus nucleotide sequence of Homo sapiens chromosome 12, GRCh38.p12 Primary Assembly (NCBI Reference Sequence: NC_000012.12).

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

An “adjuvant” refers to one or more substances that cause stimulation of the immune system. In this context, an adjuvant is used to enhance an immune response to one or more vaccine antigens. An adjuvant may be administered to a subject before, in combination with, or after administration of the vaccine. Examples of chemical compounds used as adjuvants include aluminum compounds, oils, block polymers, immune stimulating complexes, vitamins and minerals (e.g., vitamin E, vitamin A, selenium, and vitamin B12), Quil A (saponins), bacterial and fungal cell wall components (e.g., lipopolysaccarides, lipoproteins, and glycoproteins), hormones, cytokines, and co-stimulatory factors. Table 1 provides a summary of adjuvants in clinical trials.

TABLE 1
Adjuvants in clinical development

| | Company | Class | Indications | Stage |
|---|---|---|---|---|
| Montanide | Various | O/W emulsion | Malaria, cancer | Phase III |
| PLG | Novartis | Polymeric microparticles | DNA vaccine (HIV) | Phase I |
| Flagellin | Vaxinnate | Flagellin linked to antigen | Flu | Phase I |
| Q [symbol] 21 | Antigenics | Saponin | Various | Phase I |
| A [symbol] 01 | GSK | MPL + liposome + QS21 | Malaria, TB | Phase II |
| A [symbol] 02 | GSK | MPL + W/O emulsion + QS21 | Malaria | Phase II |
| A [symbol] 15 | GSK | AS01 + CpG | breast cancer | Phase I |
| [symbol] C529 | Dynavax | Synthetic MPL + [symbol] | HBV | Phase II |
| L-BLP25 | Merck | Liposomes + MPL | NSCLC | Phase III |
| [symbol] scom | CSL, Isconov [symbol] | Saponins + cholesterol + | Various | Phase I |
| | | phospholipids | | |
| IC3 [symbol] | [symbol] ntercell | Peptide + oligonucleotide | TB | Phase I |
| CpG | Coley/Pfizer, | Oligonucleotide + alum, | HBV, malaria, | |
| | Novartis, | oligonucluotide + | HVC, cancer | |
| | [symbol] dera | MF59, oligonucleotide | | |
| MF59 + MTP-PE | Chiron/Novartis | Lipidated MDP + O/W emulsion | HIV, Flu | Phase I |
| ISS | Dynavax | Oligonucleotide alum | HBV | Phase II |

[symbol]

indicates data missing or illegible when filed

Distinct adjuvants differ in their ability to induce helper T (Th) cell functions. MF59, ISCOMs, Toll-like receptor 2 (TLR2) and TLR5 ligands enhance T cell and antibody responses without altering their Th1/Th2 cell balance. In contrast, more polarized Th1 cell responses are induced by agonists of TLR3, TLR4, TLR7-TLR8, and TLR9. Complete Freund's adjuvant (CFA) and CAF01 induce mixed Th1 and Th17 cell responses. To induce CD8+ T cells, a vaccine has to engage the MHC class I processing pathway, triggering dendritic cell (DC) activation and inducing type-I interferon (IFN) production.

The term “adapter” refers to a short, chemically synthesized, nucleic acid sequence which can be used to ligate to the end of a nucleic acid sequence in order to facilitate attachment to another molecule. The adapter can be single-stranded or double-stranded. An adapter can incorporate a short (typically less than 50 base pairs) sequence useful for PCR amplification or sequencing.

As used herein, the terms “amplify” or “amplification” with respect to nucleic acid sequences, refer to methods that increase the representation of a population of nucleic acid sequences in a sample. Nucleic acid amplification methods are well known to the skilled artisan and include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step amplifications, rolling circle amplification (RCA), recombinase-polymerase amplification (RPA) (TwistDx, Cambridge, UK), transcription mediated amplification, signal mediated amplification of RNA technology, loop-mediated isothermal amplification of DNA, helicase-dependent amplification, single primer isothermal amplification, and self-sustained sequence replication (3SR), including multiplex versions or combinations thereof. Copies of a particular nucleic acid sequence generated in vitro in an amplification reaction are called “amplicons” or “amplification products.”

As used herein, an “antigen” refers to a molecule to which an antibody can selectively bind. The antigen may be a protein, carbohydrate, nucleic acid, lipid, hapten, or other naturally occurring or synthetic compound. However, some antigens fail to elicit antibody production by themselves. Antigens that are capable of inducing antibody production on their own are referred to as “immunogens.”

The terms “cancer” or “tumor” are used interchangeably and refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. As used herein, the term “cancer” includes premalignant, as well as malignant cancers. Examples of cancers include, but are not limited to, neuroblastoma, melanoma, non-Hodgkin's lymphoma, Epstein-Barr related lymphoma, Hodgkin's lymphoma, retinoblastoma, small cell lung cancer, brain tumors, leukemia, epidermoid carcinoma, prostate cancer, renal cell carcinoma, transitional cell carcinoma, breast cancer, ovarian cancer, lung cancer colon cancer, liver cancer, stomach cancer, and other gastrointestinal cancers.

As used herein, a “carrier” is an exogenous protein to which small, non-immunogenic or poorly immunogenic antigens (e.g., haptens) can be conjugated to so as to enhance the immunogenicity of the antigens. Examples of such carriers include keyhole limpet hemocyanin (KLH), serum globulins, serum albumins, ovalbumins, and the like.

The terms “complementary” or “complementarity” as used herein with reference to polynucleotides (i.e., a sequence of nucleotides such as an oligonucleotide or a target nucleic acid) refer to the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence, and can also be a cDNA.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease or condition, a positive control (a compound or composition known to exhibit the desired therapeutic effect) and a negative control (a subject or a sample that does not receive the therapy or receives a placebo) are typically employed.

A “control nucleic acid sample” or “reference nucleic acid sample” as used herein, refers to nucleic acid molecules from a control or reference sample. In certain embodiments, the reference or control nucleic acid sample is a wild type or a non-mutated DNA or RNA sequence. In certain embodiments, the reference nucleic acid sample is purified or isolated (e.g., it is removed from its natural state). In other embodiments, the reference nucleic acid sample is from a non-tumor sample, e.g., a blood control, a normal adjacent tumor (NAT), or any other non-cancerous sample from the same or a different subject.

“Detecting” as used herein refers to determining the presence of a mutation or polymorphism in a nucleic acid of interest in a sample. Detection does not require the method to provide 100% sensitivity. Analysis of nucleic acid markers can be performed using techniques known in the art including, but not limited to, sequence analysis, and electrophoretic analysis. Non-limiting examples of sequence analysis include Maxam-Gilbert sequencing, Sanger sequencing, capillary array DNA sequencing, thermal cycle sequencing (Sears et al., Biotechniques, 13:626-633 (1992)), solid-phase sequencing (Zimmerman et al., Methods Mol. Cell Biol, 3:39-42 (1992)), sequencing with mass spectrometry such as matrix-assisted laser desorption/ionization time-of-flight mass spectrometry (MALDI-TOF/MS; Fu et al., Nat. Biotechnol, 16:381-384 (1998)), and sequencing by hybridization. Chee et al., Science, 274:610-614 (1996); Drmanac et al., Science, 260:1649-1652 (1993); Drmanac et al., Nat. Biotechnol, 16:54-58 (1998). Non-limiting examples of electrophoretic analysis include slab gel electrophoresis such as agarose or polyacrylamide gel electrophoresis, capillary electrophoresis, and denaturing gradient gel electrophoresis. Additionally, next generation sequencing methods can be performed using commercially available kits and instruments from companies such as the Life Technologies/Ion Torrent PGM or Proton, the Illumina HiSEQ or MiSEQ, and the Roche/454 next generation sequencing system.

“Detectable label” as used herein refers to a molecule or a compound or a group of molecules or a group of compounds used to identify a nucleic acid or protein of interest. In some embodiments, the detectable label may be detected directly. In other embodiments, the detectable label may be a part of a binding pair, which can then be subsequently detected. Signals from the detectable label may be detected by various means and will depend on the nature of the detectable label. Detectable labels may be isotopes, fluorescent moieties, colored substances, and the like. Examples of means to detect detectable labels include but are not limited to spectroscopic, photochemical, biochemical, immunochemical, electromagnetic, radiochemical, or chemical means, such as fluorescence, chemifluorescence, or chemiluminescence, or any other appropriate means.

As used herein, the term “effective amount” refers to a quantity sufficient to achieve a desired therapeutic and/or prophylactic effect, e.g., an amount which results in the prevention of, or a decrease in a disease or disorder or one or more signs or symptoms associated with a disease or disorder. In the context of therapeutic or prophylactic applications, the amount of a composition administered to the subject will depend on the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compounds may be administered to a subject having one or more signs or symptoms of a disease or disorder. As used herein, a “therapeutically effective amount” of a compound refers to compound levels in which the physiological effects of a disease or disorder are, at a minimum, ameliorated.

“Gene” as used herein refers to a DNA sequence that comprises regulatory and coding sequences necessary for the production of an RNA, which may have a non-coding function (e.g., a ribosomal or transfer RNA) or which may include a polypeptide or a polypeptide precursor. The RNA or polypeptide may be encoded by a full length coding sequence or by any portion of the coding sequence so long as the desired activity or function is retained. Although a sequence of the nucleic acids may be shown in the form of DNA, a person of ordinary skill in the art recognizes that the corresponding RNA sequence will have a similar sequence with the thymine being replaced by uracil, i.e., “T” is replaced with “U.”

As used herein, the term “hapten” refers to a non-immunogenic or poorly immunogenic molecule that can selectively bind to an antibody, but cannot induce an adaptive immune response on its own. Haptens must be chemically linked to protein carriers to elicit antibody and T cell responses.

As used herein, “higher order conformation” refers to the three-dimensional shape formed by two or more glucan molecules interacting with one another and establishing relatively stable interchain associations through hydrogen bonds.

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Hybridizations are typically and preferably conducted with probe-length nucleic acid molecules, preferably 15-100 nucleotides in length, more preferably 18-50 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tₘ) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

As used herein, “immune response” refers to the action of one or more of lymphocytes, antigen presenting cells, phagocytic cells, granulocytes, and soluble macromolecules produced by the aforementioned cells or the liver or spleen (including antibodies, cytokines, and complement) that results in selective damage to, destruction of, or elimination from the human body of cancerous cells, metastatic tumor cells, infectious pathogens etc. An immune response may include a cellular response, such as a T-cell response that is an alteration (modulation, e.g., significant enhancement, stimulation, activation, impairment, or inhibition) of cellular, i.e., T-cell function. An immune response may also include humoral (antibody) response.

As used herein, the terms “individual”, “patient”, or “subject” are used interchangeably and refer to an individual organism, a vertebrate, a mammal, or a human. In a preferred embodiment, the individual, patient or subject is a human.

As used herein, the term “library” refers to a collection of nucleic acid sequences, e.g., a collection of nucleic acids derived from whole genomic, subgenomic fragments, cDNA, cDNA fragments, RNA, RNA fragments, or a combination thereof. In one embodiment, a portion or all of the library nucleic acid sequences comprises an adapter sequence. The adapter sequence can be located at one or both ends. The adapter sequence can be useful, e.g., for a sequencing method (e.g., an NGS method), for amplification, for reverse transcription, or for cloning into a vector.

The library can comprise a collection of nucleic acid sequences, e.g., a target nucleic acid sequence (e.g., a tumor nucleic acid sequence), a reference nucleic acid sequence, or a combination thereof. In some embodiments, the nucleic acid sequences of the library can be derived from a single subject. In other embodiments, a library can comprise nucleic acid sequences from more than one subject (e.g., 2, 3, 4, 5, 6, 7, 8, 9, 10, 20, 30 or more subjects). In some embodiments, two or more libraries from different subjects can be combined to form a library having nucleic acid sequences from more than one subject.

A “library nucleic acid sequence” refers to a nucleic acid molecule, e.g., a DNA, RNA, or a combination thereof, that is a member of a library. Typically, a library nucleic acid sequence is a DNA molecule, e.g., genomic DNA or cDNA. In some embodiments, a library nucleic acid sequence is fragmented, e.g., sheared or enzymatically prepared, genomic DNA. In certain embodiments, the library nucleic acid sequences comprise sequence from a subject and sequence not derived from the subject, e.g., adapter sequence, a primer sequence, or other sequences that allow for identification, e.g., “barcode” sequences.

“Next-generation sequencing or NGS” as used herein, refers to any sequencing method that determines the nucleotide sequence of either individual nucleic acid molecules (e.g., in single molecule sequencing) or clonally expanded proxies for individual nucleic acid molecules in a high throughput parallel fashion (e.g., greater than 10³, 10⁴, 10⁵ or more molecules are sequenced simultaneously). In one embodiment, the relative abundance of the nucleic acid species in the library can be estimated by counting the relative number of occurrences of their cognate sequences in the data generated by the sequencing experiment. Next generation sequencing methods are known in the art, and are described, e.g., in Metzker, M. Nature Biotechnology Reviews 11:31-46 (2010).

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. Oligonucleotides of the method which function as primers or probes are generally at least about 10-15 nucleotides long and more preferably at least about 15 to 25 nucleotides long, although shorter or longer oligonucleotides may be used in the method. The exact size will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

As used herein, the term “overall survival” or “OS” means the observed length of life from the start of treatment to death or the date of last contact.

As used herein, “pattern recognition receptors” or “PRRs” refer to binding molecules that recognize molecular structures common to large groups of microbes. PRRs include TLRs recognizing lipids, lipoproteins, nucleic acids, and proteins; NOD-like receptors (NLR, “nucleotide-binding domain and leucine-rich repeat containing” receptors) responding to peptidoglycan species, flagellin, toxins, and ATP; helicases (RIG-I-like receptors, RLR) triggered by cytoplasmic RNA; and C-type lectin receptors (CLRs) recognizing carbohydrates and lipids. PRRs signal through pathways involving distinct adaptors and intermediates, such as MyD88, TRIF, RIP2, CARDS, and IPS-1 that partially dictate the outcome of receptor-ligand interaction. Two key transcriptional programs involving the transcription factors NF-kB, IRF-3, and IRF-7 are activated by these signaling circuits, resulting in the induction of genes encoding cytokines, chemokines, and costimulatory molecules that play a key role in priming, expansion, and polarization of immune responses.

As used herein, the term “polypeptide,” means a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art.

As used herein, the term “poorly immunogenic antigen” refers to an antigen that does not elicit a protective or therapeutically effective response in a patient, e.g., an antigen that does not induce an immune response that is sufficient to treat or prevent a disease or condition described herein or one or more signs or symptoms associated with a disease or condition described herein.

As used herein, “prevention” or “preventing” of a disease or medical condition refers to a compound that, in a statistical sample, reduces the occurrence of the disease or medical condition in the treated sample relative to an untreated control sample, or delays the onset of one or more symptoms of the disease or medical condition relative to the untreated control sample.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of dsDNA. A “reverse primer” anneals to the sense-strand of dsDNA.

As used herein, “primer pair” refers to a forward and reverse primer pair (i.e., a left and right primer pair) that can be used together to amplify a given region of a nucleic acid of interest.

“Probe” as used herein refers to nucleic acid that interacts with a target nucleic acid via hybridization. A probe may be fully complementary to a target nucleic acid sequence or partially complementary. The level of complementarity will depend on many factors based, in general, on the function of the probe. A probe or probes can be used, for example to detect the presence or absence of a mutation or polymorphism in a nucleic acid sequence by virtue of the sequence characteristics of the target. Probes can be labeled or unlabeled, or modified in any of a number of ways well known in the art. A probe may specifically hybridize to a target nucleic acid. Probes may be DNA, RNA or a RNA/DNA hybrid. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases, modified sugar moieties, and modified internucleotide linkages. A probe may be used to detect the presence or absence of a target nucleic acid. Probes are typically at least about 10, 15, 20, 25, 30, 35, 40, 50, 60, 75, 100 nucleotides or more in length.

As used herein, “progression free survival” or “PFS” is the time from treatment to the date of the first confirmed disease progression per RECIST 1.1 criteria.

“RECIST” shall mean an acronym that stands for “Response Evaluation Criteria in Solid Tumors” and is a set of published rules that define when cancer patients improve (“respond”), stay the same (“stable”) or worsen (“progression”) during treatments. Response as defined by RECIST criteria have been published, for example, at Journal of the National Cancer Institute, Vol. 92, No. 3, Feb. 2, 2000 and RECIST criteria can include other similar published definitions and rule sets. One skilled in the art would understand definitions that go with RECIST criteria, as used herein, such as “Partial Response (PR),” “Complete Response (CR),” “Stable Disease (SD)” and “Progressive Disease (PD).” As used herein, “CR2” refers to a patient that has achieved CR, relapsed and achieved CR again.

As used herein, a “sample” or “biological sample” refers to a substance that is being assayed for the presence of a mutation or polymorphism in a nucleic acid of interest. Processing methods to release or otherwise make available a nucleic acid for detection are well known in the art and may include steps of nucleic acid manipulation. A biological sample may be a body fluid or a tissue sample isolated from a subject. In some cases, a biological sample may consist of or comprise whole blood, platelets, red blood cells, white blood cells, plasma, sera, urine, feces, epidermal sample, vaginal sample, skin sample, cheek swab, sperm, amniotic fluid, cultured cells, bone marrow sample, tumor biopsies, aspirate and/or chorionic villi, cultured cells, endothelial cells, synovial fluid, lymphatic fluid, ascites fluid, interstitial or extracellular fluid and the like. The term “sample” may also encompass the fluid in spaces between cells, including gingival crevicular fluid, bone marrow, cerebrospinal fluid (CSF), saliva, mucus, sputum, semen, sweat, urine, or any other bodily fluids. Samples can be obtained from a subject by any means including, but not limited to, venipuncture, excretion, ejaculation, massage, biopsy, needle aspirate, lavage, scraping, surgical incision, or intervention or other means known in the art. A blood sample can be whole blood or any fraction thereof, including blood cells (red blood cells, white blood cells or leucocytes, and platelets), serum and plasma. Fresh, fixed or frozen tissues may also be used. In one embodiment, the sample is preserved as a frozen sample or as formaldehyde- or paraformaldehyde-fixed paraffin-embedded (FFPE) tissue preparation. For example, the sample can be embedded in a matrix, e.g., an FFPE block or a frozen sample. Whole blood samples of about 0.5 to 5 ml collected with EDTA, ACD or heparin as anti-coagulant are suitable.

The term “sensitivity,” as used herein in reference to the methods of the present technology, is a measure of the ability of a method to detect a preselected sequence variant in a heterogeneous population of sequences. A method has a sensitivity of S % for variants of F % if, given a sample in which the preselected sequence variant is present as at least F % of the sequences in the sample, the method can detect the preselected sequence at a preselected confidence of C %, S % of the time. By way of example, a method has a sensitivity of 90% for variants of 5% if, given a sample in which the preselected variant sequence is present as at least 5% of the sequences in the sample, the method can detect the preselected sequence at a preselected confidence of 99%, 9 out of 10 times (F=5%; C=99%; S=90%).

As used herein, the term “separate” therapeutic use refers to an administration of at least two active ingredients at the same time or at substantially the same time by different routes.

As used herein, the term “sequential” therapeutic use refers to administration of at least two active ingredients at different times. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “seroconversion” refers to the development of detectable antibodies that are directed against a specific target antigen in the blood or serum of a subject as a result of infection or immunization.

As used herein, the term “simultaneous” therapeutic use refers to the administration of at least two active ingredients by the same route and at the same time or at substantially the same time.

As used herein, the term “single nucleotide polymorphism” or “SNP” are DNA sequence variations that occur when a single nucleotide (A,T,C, or G) in a gene sequence is changed. SNPs can occur every 100 to 300 bases along the human genome and may be associated with a disease state. A SNP includes all single base variants, thus including nucleotide insertions and deletions in addition to single nucleotide substitutions. There are two types of nucleotide substitutions. A transition is the replacement of one purine by another purine or one pyrimidine by another pyrimidine. A transversion is the replacement of a purine for a pyrimidine, or vice versa.

As used herein, “survival” refers to the subject remaining alive, and includes overall survival as well as progression free survival.

The term “specific” as used herein in reference to an oligonucleotide primer means that the nucleotide sequence of the primer has at least 12 bases of sequence identity with a portion of the nucleic acid to be amplified when the oligonucleotide and the nucleic acid are aligned. An oligonucleotide primer that is specific for a nucleic acid is one that, under the stringent hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, at least 95% and more preferably at least 98% sequence identity.

“Specificity,” as used herein, is a measure of the ability of a method to distinguish a truly occurring preselected sequence variant from sequencing artifacts or other closely related sequences. It is the ability to avoid false positive detections. False positive detections can arise from errors introduced into the sequence of interest during sample preparation, sequencing error, or inadvertent sequencing of closely related sequences like pseudo-genes or members of a gene family. A method has a specificity of X % if, when applied to a sample set of N_{Total} sequences, in which X_{True} sequences are truly variant and X_{Not true} are not truly variant, the method selects at least X % of the not truly variant as not variant. E.g., a method has a specificity of 90% if, when applied to a sample set of 1,000 sequences, in which 500 sequences are truly variant and 500 are not truly variant, the method selects 90% of the 500 not truly variant sequences as not variant. Exemplary specificities include 90, 95, 98, and 99%.

The term “stringent hybridization conditions” as used herein refers to hybridization conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5× Denhart's solution at 42° C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

As used herein, the terms “target sequence” and “target nucleic acid sequence” refer to a specific nucleic acid sequence to be detected and/or quantified in the sample to be analyzed.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

The term “vaccine” as used herein is a preparation used to enhance protective immunity against cancer, or infectious agents such as viruses, fungi, bacteria and other pathogens. A vaccine may be useful as a prophylactic agent or a therapeutic agent. Vaccines contain cells or antigens which, when administered to the body, induce an immune response with the production of antibodies and immune lymphocytes (T-cells and B-cells).

“Whole cell tumor vaccines”, also referred to as “whole tumor vaccines” comprise tumor cells which may be autologous or allogeneic for the patient and comprise cancer antigens which can stimulate the body's immune system. Unlike the administration of an antigen-specific vaccine, a whole cell tumor vaccine exposes a large number of cancer specific (unique or up-regulated) antigens to the patient's immune system. The whole cell tumor vaccine may comprise intact cells or a cell lysate. The use of such a lysate or intact cell preparation means that the vaccine will comprise in excess of 10 antigens, typically in excess of 30 antigens. Whole cell tumor vaccines may comprise tumor cells that have been modified in vitro, e.g., irradiated and dead tumor cells or live tumor cells.

### Yeast Beta-Glucans of the Present Technology

Beta-glucans are polymers containing a backbone of beta-1,3-linked and beta-1,4-D-glucose molecules with 1,6-linked side-chains. The frequency of these side-chains regulates secondary structures and biochemical properties. Beta-glucans are found in many foods, such as mushrooms, oats, rice, barley, seaweed, baker's yeast and fungi. Glucan-containing extracts include Lentinan (from Shiitake mushroom), PSK (from Coriolus versicolor), laminarin (from seaweed), Schizophyllan, Betafectin and Maitake d-fraction. Beta-1,3-glucan is the component responsible for the majority of biological activities of zymosan, a commonly used leukocyte stimulant derived from the cell wall of Bakers' yeast (Saccharomyces cerevisiae).

Depending upon the source and method of isolation, beta-glucans have various degrees of branching and of linkages in the side chains. The frequency and hinge-structure of side chains determine its immunomodulatory effect. Beta-glucans of fungal and yeast origin are normally insoluble in water, but can be made soluble either by acid hydrolysis or derivatization by introducing charged groups like phosphate, sulfate, amine, carboxymethyl and so forth to the molecule (Seljelid R, Biosci. Rep. 6:845-851 (1986); Williams et al., Immunopharmacology 22:139-156 (1991)).

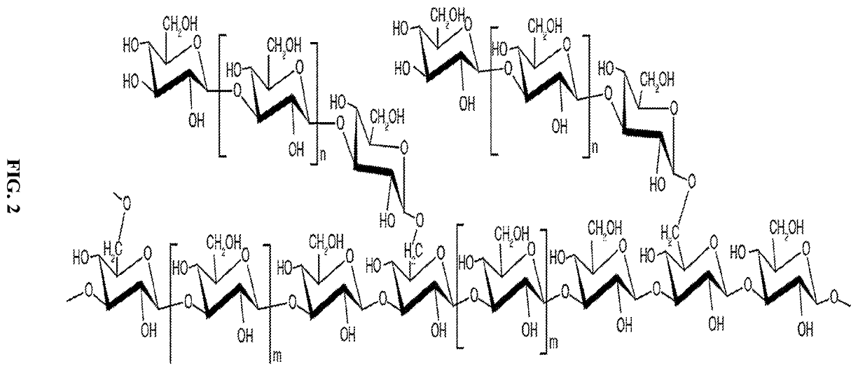

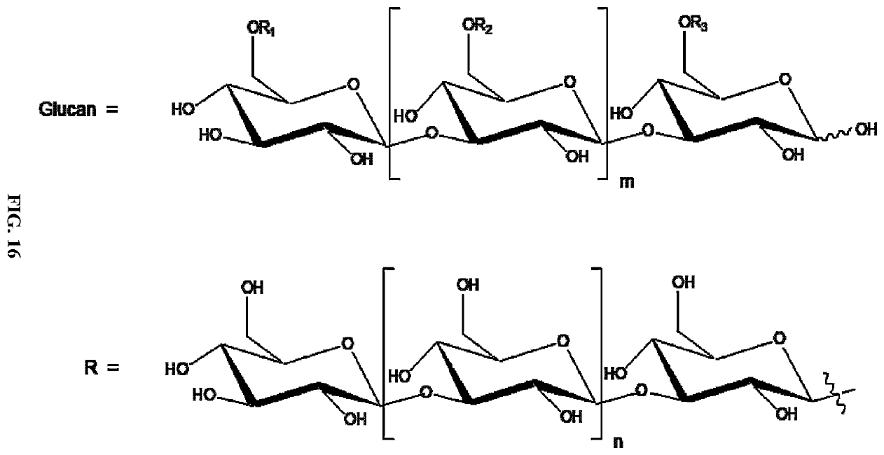

The yeast beta-glucans of the present technology comprises a plurality of β-(1,3) side chains linked to a β-(1,3) backbone via β-(1,6) linkages, and has a range of average molecular weights from about 6 kDa to about 30 kDa, from about 6 kDa to about 25 kDa, or from about 16 kDa to about 17 kDa (Biotec Pharamacon ASA, Tromso, Norway). FIG. 16 shows the generic structure of the yeast beta-glucans of the present technology. An exemplar molecular structure of the yeast beta-glucans of the present technology is provided in FIG. 2 (n is an integer from 0 to about 50, m is an integer from about 35 to about 2000).

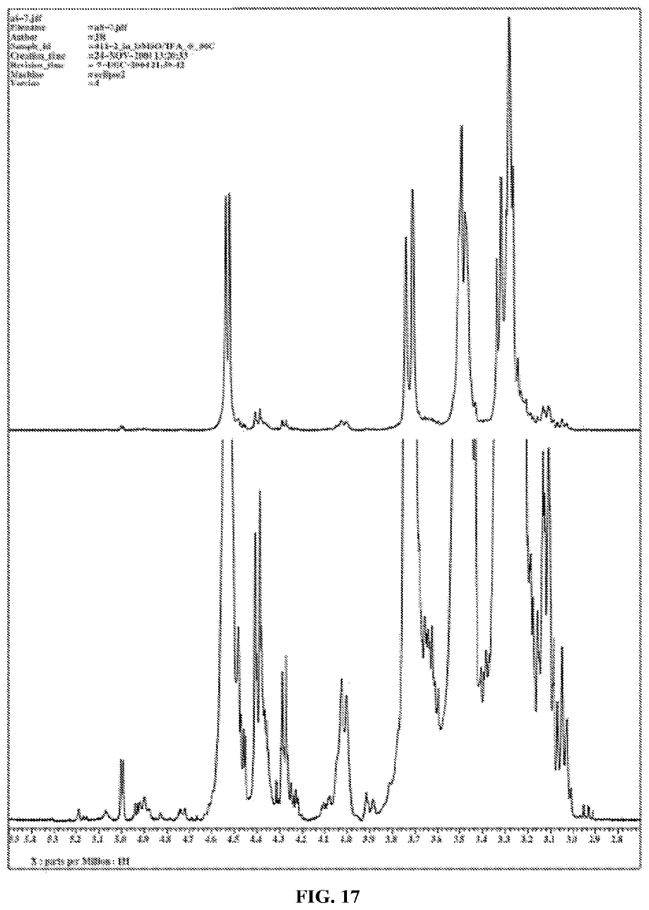

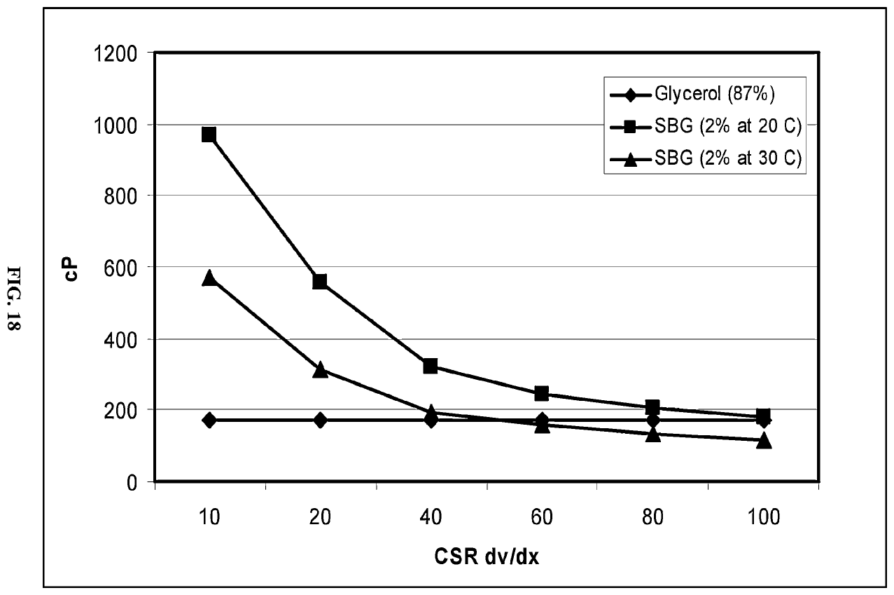

The beta-glucan molecules form a higher order conformation, resulting in gelling and high viscosity profile. The NMR profile and viscosity profile of the yeast beta-glucans of the present technology are shown in FIG. 17 and FIG. 18, respectively.

The yeast beta-glucans of the present technology are treated with a hydrolyzing agent like an acid or enzyme to significantly reduce or eliminate (1,6) linkages within the glucan branches (a single (1,6) link is required to form the branch). In some embodiments, less than 10%, less than 5%, less than 3% or less than 2% of the glycosidic bonds in the beta-glucan molecule will be (1,6) linkages. These products can be particulate, semi-soluble, soluble or a gel. In certain embodiments, production of solubilized yeast beta-glucans include the addition of formic acid to the extracted yeast beta-glucans to a final concentration of 75% w/v and heating the suspension to facilitate formolysis. An example of a soluble hydrolyzed yeast beta-glucan of the present technology is Soluble Beta Glucan (Biotec Pharmacon ASA, Tromso, Norway). Soluble Beta Glucan is an underivatized (in terms of chemical modifying groups) aqueous soluble β-1,3/1,6-glucan, characterized by NMR and chemical analysis as containing a linear β-1,3-glucan backbone having side chains of β-1,3-linked D-glucose units wherein the side chains are attached to the backbone via β-1,6-linkages, wherein the number of β-1,6 moieties in the side chains (not including at the backbone/side chain branch point) is considerably reduced as compared to the structure of said glucan in the yeast cell wall. Soluble Beta Glucan presents durable interchain associations as demonstrated by its high viscosity profile and gelling behavior (FIG. 18). A non-limiting example of such a composition is:

| | | |
|---|---|---|
| Ingredient | Range | Typical Value |

| | | | |
|---|---|---|---|
| 1,3/1,6-beta-D-glucan | 18-22 | g/kg | 20 g/kg |
| Proteins | 1 | g/kg (max) | <1 g/kg |
| Ash | 1 | g/kg (max) | <1 g/kg |
| Water | 977-983 | g/kg | 980 g/kg |

Products having the desired structural features and showing a higher order conformation like Solubilized Beta Glucan may be administered orally, intraperitoneally, subcutaneously, intra-muscularly or intravenously. Functional dose range of the glucans can be readily determined by one of ordinary skill in the art. For example, when administered orally the functional dose range would be in the area of 1-500 mg/kg/day, 10-200 mg/kg/day, or 20-80 mg/kg/day. When administered parenterally, the functional dose range may be 0.1-10 mg/kg/day.

In the present technology, a yeast beta-1,3-glucan is used in combination with a vaccine. In certain embodiments, the yeast beta-1,3-glucan is administered in the amount of 0.1-4 mg. The above mentioned pharmaceutical compositions may contain pharmaceutically acceptable carriers and other ingredients known to enhance and facilitate drug administration. The relative amounts of the active ingredient, the pharmaceutically acceptable carrier, and any additional ingredients in a pharmaceutical composition of the present technology will vary, depending upon the identity, size, and condition of the subject treated. Such a pharmaceutical composition may comprise the active ingredient alone, in a form suitable for administration to a subject, or the pharmaceutical composition may comprise the active ingredient and one or more pharmaceutically acceptable carriers, one or more additional ingredients, or any combination thereof. The active ingredient may be present in the pharmaceutical composition in forms which are generally well known in the art.

Typically, dosages of the yeast beta-glucans of the present technology administered to a subject, will vary depending upon any number of factors, including but not limited to, the type of subject and type of cancer and disease state being treated, the age of the subject, the route of administration and the relative therapeutic index. The route(s) of administration will be readily apparent to the skilled artisan and will depend upon any number of factors including the type and severity of the disease being treated, the gender and age of the patient being treated, and the like.

Formulations suitable for oral administration of the yeast beta-glucans include, but are not limited to, an aqueous or oily suspension, an aqueous or oily solution, an emulsion or a particulate formulation. Such formulations can be administered by any means including, but not limited to, soft gelatin capsules.

Liquid formulations of the yeast beta-glucans disclosed herein that are suitable for oral administration may be prepared, packaged, and sold either in liquid form or in the form of a dry product intended for reconstitution with water or other suitable vehicle prior to use. Administration can be by a variety of different routes including intravenous, subcutaneous, intranasal, buccal, transdermal and intrapulmonary. One of ordinary skill in the art would be able to determine the desirable routes of administration, and the kinds of formulations suitable for a particular route of administration.

In general, the yeast beta-glucan can be administered to a subject as frequently as several times daily, or it may be administered less frequently, such as once a day. The vaccine treatment will for instance depend upon the type of antigen, the type of cancer, the severity of the cancer, and the condition of each patient. The yeast beta-glucan treatment is closely interrelated with the vaccine treatment regimen, and could be prior to, concurrent with, or after the administration of the vaccine. The frequency of the yeast beta-glucan and vaccine dose will be readily apparent to the skilled artisan and will depend upon any number of factors, such as, but not limited to, the extent and severity of the disease being treated, and the type and age of the patients.

### Dectin-1

Dectin-1 (also known as C-type lectin domain family 7 member A (CLEC7A)) is mainly expressed on dendritic cell and macrophage surfaces, and is known for its role in antifungal immunity through its ability to recognize cell wall β-glucans. Upon activation by these carbohydrates, dectin-1 transduces intracellular signaling through several pathways including phagocytosis, the respiratory burst, neutrophil extracellular trap formation, inflammasome activation and cytokine and chemokine production. Like the Toll-like receptors (TLRs), dectin-1 instructs adaptive immunity, promoting Th1- and Th17-type responses. Dectin-1 can also synergize with other pattern recognition receptors (PRRs) to regulate innate and adaptive immunities. Recent data suggest that dectin-1 can recognize a broader range of pathogens including bacteria, as well as endogenous ligands, thus playing a role in autoimmune diseases and cancer. See Asamaphan P et al., Dectin-1 (CLEC7A, BGR, CLECSF12), in Yamasaki S (ed): C-TYPE LECTIN RECEPTORS IN IMMUNITY. Tokyo, Springer Japan, 2016, pp 51-63.

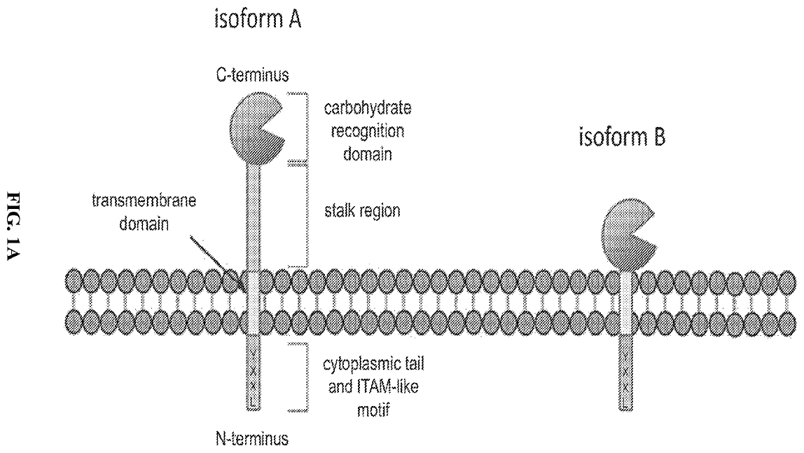

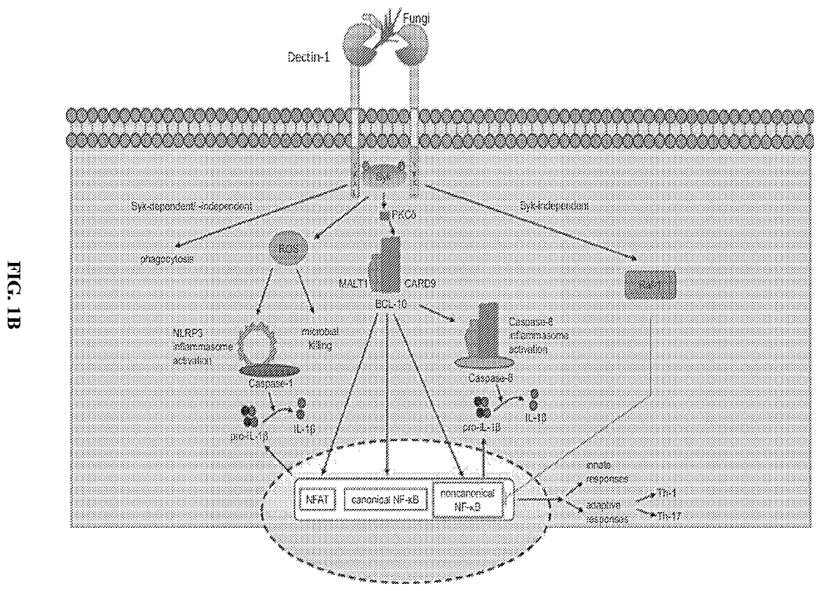

Dectin-1 is encoded by the Dectin-1 cluster within the natural killer gene complex (NKC) on chromosome 6 in mouse and chromosome 12 in Homo sapiens. This type II transmembrane receptor contains a single carbohydrate recognition domain (CRD), a stalk region, a transmembrane region and a cytoplasmic tail (FIGS. 1A-1B). The N-terminal cytoplasmic tail has an immunoreceptor tyrosine-based activation motif (ITAM)-like, YXXL, which can signal downstream. Dectin-1 has two major isoforms (full length isoform A and truncated isoform B, which is missing the stalk region) plus minor isoforms (isoform C—H). Willment et al., J Biol Chem 276:43818-23 (2001); Heinsbroek et al., J Immunol 176:5513-8 (2006). Only the two major isoforms (A and B) interact with extracellular ligands. Dectin-1 is found primarily on neutrophils, monocytes, macrophages and DCs; although it is also been found on microglia, eosinophils, mast cells and certain lymphocytes, including B cells and γδ T-cells. Dectin-1 is upregulated by granulocyte macrophage colony-stimulating factor (GM-CSF), IL-4 and IL-13, but downregulated by IL-10, LPS and dexamethasone. Dectin-1 can be induced on epithelial cells.

Dectin-1 recognizes β-1,3-glucans and fungal pathogens including Candida albicans, Aspergillus fumigatus, Coccidioides immitis and Pneumocystis carinii. The minimum unit ligand for dectin-1 is between 11 and 13 glucose monomers and the affinity of its interaction with these carbohydrates is influenced by side chain branching. Structural and mutation analyses have revealed a shallow groove on the surface of dectin-1, which may be the ligand-binding site, and that this groove is flanked by two residues, Trp221 and His223, that are indispensable for ligand binding. Dectin-1 can also interact with endogenous ligands such as intermediate filament protein, vimentin, thereby driving lipid oxidation in atherosclerosis. Dectin-1 is required for reverse transcytosis of secretory IgA-antigen complexes by intestinal M cells and the induction of subsequent mucosal and systemic antibody responses. Dectin-1 also recognizes galactosylated IgG1 through FcγRIIB, thereby inhibiting complement-mediated inflammation as well as N-glycans on the surface of tumor cells. The ITAM-like motif of dectin-1 is phosphorylated by Src kinases. This Syk-dependent signaling pathway is unusual by virtue of a single phosphorylated tyrosine residue and the requirement of receptor dimerization. Through PKC delta and the CARD9-Bcl10-Malt1 complex, induction of canonical and non-canonical NF-κB subunits (p65/c-REL and RelB, respectively) and interferon regulatory factor (IRF)1, results in gene transcription. Instead of CARD9, ERK activation through Ras-GRF1 and H-Ras can also suffice. Syk activation can also induce IRF5 and nuclear factor of activated T-cells (NFAT) through phospholipase C gamma and Calcineurin. The Syk-independent pathway uses Raf-1 to activate NF-υB. Dectin-1 receptor clustering into a ‘phagocytic synapse’ and exclusion of regulatory tyrosine phosphatases is required for effective signaling that controls cellular responses including actin-mediated phagocytosis, neutrophil extracellular trap (NET) formation, activation of the respiratory burst and DC maturation and antigen presentation, in part through the use of autophagy machinery such as light chain 3 protein (LC3). Dectin-1 also induces the production of eicosanoids, several cytokines and chemokines (such as TNF, IL-10, IL-6, IL-2, IL-23, IFN-β, CCL2, CCL3) and can modulate cytokine production induced by other PRRs. Dectin-1 activates NLRP3/caspase-1 inflammasome facilitating the release of IL-1β, upon recognition of β-glucans, in a Syk-dependent manner, or a non-canonical caspase-8 inflammasome through Syk, CARD9, MALT1 and the non-receptor tyrosine kinase Tec. The induction of phagocytosis by dectin-1 in macrophages, requires Bruton's tyrosine kinase (Btk) and Vav-1 but not Syk.

Dectin-1 has an important role in antifungal immunity for both mice and humans. A functional single nucleotide polymorphism (SNP) in human CLEC7A (Y238X, rs16910526) generates a premature stop codon, leading to a protein lacking the final ten amino acids of the carbohydrate-recognition domain. The rs16910526 polymorphism eliminates dectin-1 expression on immune cell surfaces and is linked to susceptibility to A. fumigatus, Trichophyton rubrum and C. albicans. However, the high frequency of this polymorphism in European and African populations does not correlate with disease prevalence. The CLEC7A intronic SNPs rs3901533 and rs7309123 are associated with susceptibility to invasive pulmonary disease in patients with hematologic diseases; however, the detailed mechanism remains unclear. Sainz J et al., PLOS ONE 7:e32273 (2012).

Dectin-1 controls adaptive immune immunity through Th1 and Th17; Th1 for the control of systemic fungal infections and Th17 for controlling fungal infections at the mucosa. Dectin-1 is required for Th17 polarization during mucocutaneous infections with C. albicans, probably involving MALT1-dependent activation of the NF-κB subunit c-REL, required for the induction of polarizing cytokines, such as IL-1β and IL-23p19. Expression of dectin-1 on lymphocytes, such as γδ T-cells, provides an important innate source for the rapid production of IL-17 and other key cytokines during infection. Dectin-1 can induce humoral responses, stimulate cytotoxic T-cell responses and induce Th17 cells in response to some fungi, such as Paracoccidioides brasiliensis. Triggering dectin-1 can induce innate memory (or trained immunity) through the epigenetic reprogramming of monocytes and the induction of neutrophilic myeloid-derived suppressor cells. Fungi are known to evolve with mechanisms to avoid dectin-1 recognition, e.g. concealing with surface conidial hydrophobic layer and cell wall galactosaminogalactan mask of A. fumigatus, changes in cell wall structure by switching from yeast to hyphae in C. albicans, active masking of β-glucans by switching to α-glucan following infection with Histoplasma capsulatum. Intact Cladosporium cladosporioides (C. cladosporioides) exposes little of its β-glucans at the surface in order to polarize from a Th17 to an allergic Th2 response. Beyond antifungal immunity, dectin-1 has been shown to recognize Haemophilus influenzae, Salmonella typhimurium, Mycobacterium tuberculosis and Leishmania infantum. Optimal immune responses to fungi requires synergistic signaling through dectin-1 and TLRs to induce cytokines, such as TNF and IL-23, while repressing others, such as IL-12. For DCs, dectin-1 alone is sufficient for the production of TNF-α; for macrophages, co-stimulation with TLRs is required. Optimal Th17 response to C. albicans required signaling from both dectin-1 and dectin-2. Dectin-1 in combination with TLR2 could amplify mannose receptor-induced IL-17 production. Dectin-1 interacts with tetraspanins CD63 and CD37, which regulate the surface expression and functional responses of dectin-1. Engagement of the CLR mincle during chromoblastomycoses promoted non-protective Th2 immunity by suppressing dectin-1-mediated Th1 responses.

### Methods for Detecting Polynucleotides Associated with Enhanced Antibody Response to Vaccines When Using β-Glucan as an Adjuvant

Polynucleotides associated with enhanced antibody response to the combination of vaccines and β-glucan may be detected by a variety of methods known in the art. Non-limiting examples of detection methods are described below. The detection assays in the methods of the present technology may include purified or isolated DNA or the detection step may be performed directly from a biological sample without the need for further DNA purification/isolation.

### Nucleic Acid Amplification and/or Detection

Polynucleotides associated with enhanced immunogenic response to vaccines when using β-glucan as a vaccine adjuvant can be detected by the use of nucleic acid amplification techniques that are well known in the art. The starting material may be genomic DNA, cDNA, RNA or mRNA. Nucleic acid amplification can be linear or exponential. Specific polymorphisms, variants or mutations may be detected by the use of amplification methods with the aid of oligonucleotide primers or probes designed to interact with or hybridize to a particular target sequence in a specific manner, thus amplifying only the target variant.

Non-limiting examples of nucleic acid amplification techniques include polymerase chain reaction (PCR), real-time quantitative PCR (qPCR), digital PCR (dPCR), reverse transcriptase polymerase chain reaction (RT-PCR), nested PCR, ligase chain reaction (see Abravaya, K. et al., Nucleic Acids Res. (1995), 23:675-682), branched DNA signal amplification (see Urdea, M. S. et al., AIDS (1993), 7(suppl 2):S11-S14), amplifiable RNA reporters, Q-beta replication, transcription-based amplification, boomerang DNA amplification, strand displacement activation, cycling probe technology, isothermal nucleic acid sequence based amplification (NASBA) (see Kievits, T. et al., J Virological Methods (1991), 35:273-286), Invader Technology, next-generation sequencing technology or other sequence replication assays or signal amplification assays.

Primers: Oligonucleotide primers for use in amplification methods can be designed according to general guidance well known in the art as described herein, as well as with specific requirements as described herein for each step of the particular methods described. In some embodiments, oligonucleotide primers for DNA synthesis and PCR are 10 to 100 nucleotides in length, preferably between about 15 and about 60 nucleotides in length, more preferably 25 and about 50 nucleotides in length, and most preferably between about 25 and about 40 nucleotides in length.

Tₘ of a polynucleotide affects its hybridization to another polynucleotide (e.g., the annealing of an oligonucleotide primer to a template polynucleotide). In certain embodiments of the disclosed methods, the oligonucleotide primer used in various steps selectively hybridizes to a target template or polynucleotides derived from the target template (e.g., first and second strand cDNAs and amplified products). Typically, selective hybridization occurs when two polynucleotide sequences are substantially complementary (at least about 65% complementary over a stretch of at least 14 to 25 nucleotides, preferably at least about 75%, more preferably at least about 90% complementary). See Kanehisa, M., Polynucleotides Res. (1984), 12:203, incorporated herein by reference. As a result, it is expected that a certain degree of mismatch at the priming site is tolerated. Such mismatch may be small, such as a mono-, di- or tri-nucleotide. In certain embodiments, 100% complementarity exists.

Probes: Probes are capable of hybridizing to at least a portion of the nucleic acid of interest or a reference nucleic acid (i.e., wild-type sequence). Probes may be an oligonucleotide, artificial chromosome, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may be used for detecting and/or capturing/purifying a nucleic acid of interest.

Typically, probes can be about 10 nucleotides, about 20 nucleotides, about 25 nucleotides, about 30 nucleotides, about 35 nucleotides, about 40 nucleotides, about 50 nucleotides, about 60 nucleotides, about 75 nucleotides, or about 100 nucleotides long. However, longer probes are possible. Longer probes can be about 200 nucleotides, about 300 nucleotides, about 400 nucleotides, about 500 nucleotides, about 750 nucleotides, about 1,000 nucleotides, about 1,500 nucleotides, about 2,000 nucleotides, about 2,500 nucleotides, about 3,000 nucleotides, about 3,500 nucleotides, about 4,000 nucleotides, about 5,000 nucleotides, about 7,500 nucleotides, or about 10,000 nucleotides long.

Probes may also include a detectable label or a plurality of detectable labels. The detectable label associated with the probe can generate a detectable signal directly. Additionally, the detectable label associated with the probe can be detected indirectly using a reagent, wherein the reagent includes a detectable label, and binds to the label associated with the probe.

In some embodiments, detectably labeled probes can be used in hybridization assays including, but not limited to Northern blots, Southern blots, microarray, dot or slot blots, and in situ hybridization assays such as fluorescent in situ hybridization (FISH) to detect a target nucleic acid sequence within a biological sample. Certain embodiments may employ hybridization methods for measuring expression of a polynucleotide gene product. Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif, 1987); Young and Davis, PNAS. 80: 1194 (1983).

Detectably labeled probes can also be used to monitor the amplification of a target nucleic acid sequence. In some embodiments, detectably labeled probes present in an amplification reaction are suitable for monitoring the amount of amplicon(s) produced as a function of time. Examples of such probes include, but are not limited to, the 5′- exonuclease assay (TAQMAN® probes described herein (see also U.S. Pat. No. 5,538,848) various stem-loop molecular beacons (see for example, U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, for example, Kubista et al., 2001, SPIE 4264:53-58), non-FRET probes (see, for example, U.S. Pat. No. 6,150,097), Sunrise®/Amplifluor™ probes (U.S. Pat. No. 6,548,250), stem-loop and duplex Scorpion probes (Solinas et al., 2001, Nucleic Acids Research 29:E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471 ; Whitcombe et al., 1999, Nature Biotechnology. 17:804-807; Isacsson et al., 2000, Molecular Cell Probes. 14:321-328; Svanvik et al., 2000, Anal Biochem. 281 :26-35; Wolffs et al., 2001, Biotechniques 766:769-771 ; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem. Res. Toxicol. 15:118-126; and Yu et al., 2001, J. Am. Chem. Soc 14:11155-11161.

In some embodiments, the detectable label is a fluorophore. Suitable fluorescent moieties include but are not limited to the following fluorophores working individually or in combination: 4-acetamido-4′-isothiocyanatostilbene-2,2′disulfonic acid; acridine and derivatives: acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2-aminoethyl)aminonaphthalene-1-sulfonic acid (EDANS); 4-amino-N[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120),7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′- isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)amino fluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescem (TET); fiuorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron®Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; tetramethyl rhodamine isothiocyanate (TRITC); and VIC®. Detector probes can also comprise sulfonate derivatives of fluorescenin dyes with S03 instead of the carboxylate group, phosphoramidite forms of fluorescein, phosphoramidite forms of CY 5 (commercially available for example from Amersham).

Detectably labeled probes can also include quenchers, including without limitation black hole quenchers (Biosearch), Iowa Black (IDT), QSY quencher (Molecular Probes), and Dabsyl and Dabcel sulfonate/carboxylate Quenchers (Epoch).

Detectably labeled probes can also include two probes, wherein for example a fluorophore is on one probe, and a quencher is on the other probe, wherein hybridization of the two probes together on a target quenches the signal, or wherein hybridization on the target alters the signal signature via a change in fluorescence.

In some embodiments, interchelating labels such as ethidium bromide, SYBR® Green I (Molecular Probes), and PicoGreen® (Molecular Probes) are used, thereby allowing visualization in real-time, or at the end point, of an amplification product in the absence of a detector probe. In some embodiments, real-time visualization may involve the use of both an intercalating detector probe and a sequence-based detector probe. In some embodiments, the detector probe is at least partially quenched when not hybridized to a complementary sequence in the amplification reaction, and is at least partially unquenched when hybridized to a complementary sequence in the amplification reaction.

In some embodiments, the amount of probe that gives a fluorescent signal in response to an excited light typically relates to the amount of nucleic acid produced in the amplification reaction. Thus, in some embodiments, the amount of fluorescent signal is related to the amount of product created in the amplification reaction. In such embodiments, one can therefore measure the amount of amplification product by measuring the intensity of the fluorescent signal from the fluorescent indicator.

Primers or probes may be designed to selectively hybridize to a CLEC7A (dectin-1) nucleic acid sequence comprising a wild-type or a mutant rs3901533 SNP. In some embodiments, the wild-type rs3901533 SNP comprises the nucleic acid sequence 5′ GGGGATCTCTTTAAACTCCTATAGAATTTATTGCTAATGCCAGATATTTAA 3′ (SEQ ID NO: 1) or CGGGGATCTCTTTAAACTCCTATAG[A]ATTTATTGCTAATGCCAGATATTTA (SEQ ID NO: 3). In certain embodiments, the mutant rs3901533 SNP comprises the nucleic acid sequence 5′ GGGGATCTCTTTAAACTCCTATAGCATTTATTGCTAATGCCAGATATTTAA 3′ (SEQ ID NO: 2) or CGGGGATCTCTTTAAACTCCTATAG[C]ATTTATTGCTAATGCCAGATATTTA (SEQ ID NO: 4).

Additionally or alternatively, in some embodiments, the probes comprise a nucleic acid sequence of SEQ ID NO: 1, SEQ ID NO: 2, SEQ ID NO: 3, or SEQ ID NO: 4, optionally wherein the probes are detectably labelled.

Additionally or alternatively, in some embodiments, primers comprise a forward primer and a reverse primer, wherein the forward primer hybridizes toward the 5′ end of the wild-type or a mutant rs3901533 SNP and wherein the reverse primer hybridizes toward the 3′ end of the wild-type or a mutant rs3901533 SNP.

Primers or probes can be designed so that they hybridize under stringent conditions to nucleotide sequences comprising a mutant rs3901533 SNP, but not to the respective wild-type nucleotide sequences. Primers or probes can also be prepared that are complementary and specific for nucleotide sequences comprising a wild-type rs3901533 SNP, but not to any one of the corresponding mutant nucleotide sequences. Alternatively, primers or probes can be designed so that they selectively hybridize to a target nucleic acid sequence comprising SEQ ID NO: 1 or SEQ ID NO: 2. Additionally or alternatively, in some embodiments, the probes comprise (a) the 25^{th} base and (b) at least 10 contiguous bases in SEQ ID NO: 1 or SEQ ID NO: 2.

In some embodiments, detection can occur through any of a variety of mobility dependent analytical techniques based on the differential rates of migration between different nucleic acid sequences. Exemplary mobility-dependent analysis techniques include electrophoresis, chromatography, mass spectroscopy, sedimentation, gradient centrifugation, field-flow fractionation, multi-stage extraction techniques, and the like. In some embodiments, mobility probes can be hybridized to amplification products, and the identity of the target nucleic acid sequence determined via a mobility dependent analysis technique of the eluted mobility probes, as described in Published PCT Applications WO04/46344 and WO01/92579. In some embodiments, detection can be achieved by various microarrays and related software such as the Applied Biosystems Array System with the Applied Biosystems 1700 Chemiluminescent Microarray Analyzer and other commercially available array systems available from Affymetrix, Agilent, Illumina, and Amersham Biosciences, among others (see also Gerry et al., J. Mol. Biol. 292:251-62, 1999; De Bellis et al., Minerva Biotec 14:247-52, 2002; and Stears et al., Nat. Med. 9:14045, including supplements, 2003).

It is also understood that detection can comprise reporter groups that are incorporated into the reaction products, either as part of labeled primers or due to the incorporation of labeled dNTPs during an amplification, or attached to reaction products, for example but not limited to, via hybridization tag complements comprising reporter groups or via linker arms that are integral or attached to reaction products. In some embodiments, unlabeled reaction products may be detected using mass spectrometry.

### NGS Platforms

In some embodiments, high throughput, massively parallel sequencing employs sequencing-by-synthesis with reversible dye terminators. In other embodiments, sequencing is performed via sequencing-by-ligation. In yet other embodiments, sequencing is single molecule sequencing. Examples of Next Generation Sequencing techniques include, but are not limited to pyrosequencing, Reversible dye-terminator sequencing, SOLiD sequencing, Ion semiconductor sequencing, Helioscope single molecule sequencing etc.

The Ion Torrent™ (Life Technologies, Carlsbad, CA) amplicon sequencing system employs a flow-based approach that detects pH changes caused by the release of hydrogen ions during incorporation of unmodified nucleotides in DNA replication. For use with this system, a sequencing library is initially produced by generating DNA fragments flanked by sequencing adapters. In some embodiments, these fragments can be clonally amplified on particles by emulsion PCR. The particles with the amplified template are then placed in a silicon semiconductor sequencing chip. During replication, the chip is flooded with one nucleotide after another, and if a nucleotide complements the DNA molecule in a particular microwell of the chip, then it will be incorporated. A proton is naturally released when a nucleotide is incorporated by the polymerase in the DNA molecule, resulting in a detectable local change of pH. The pH of the solution then changes in that well and is detected by the ion sensor. If homopolymer repeats are present in the template sequence, multiple nucleotides will be incorporated in a single cycle. This leads to a corresponding number of released hydrogens and a proportionally higher electronic signal.

The 454™ GS FLX™ sequencing system (Roche, Germany), employs a light-based detection methodology in a large-scale parallel pyrosequencing system. Pyrosequencing uses DNA polymerization, adding one nucleotide species at a time and detecting and quantifying the number of nucleotides added to a given location through the light emitted by the release of attached pyrophosphates. For use with the 454™ system, adapter-ligated DNA fragments are fixed to small DNA-capture beads in a water-in-oil emulsion and amplified by PCR (emulsion PCR). Each DNA-bound bead is placed into a well on a picotiter plate and sequencing reagents are delivered across the wells of the plate. The four DNA nucleotides are added sequentially in a fixed order across the picotiter plate device during a sequencing run. During the nucleotide flow, millions of copies of DNA bound to each of the beads are sequenced in parallel. When a nucleotide complementary to the template strand is added to a well, the nucleotide is incorporated onto the existing DNA strand, generating a light signal that is recorded by a CCD camera in the instrument.

Sequencing technology based on reversible dye-terminators: DNA molecules are first attached to primers on a slide and amplified so that local clonal colonies are formed. Four types of reversible terminator bases (RT-bases) are added, and non-incorporated nucleotides are washed away. Unlike pyrosequencing, the DNA can only be extended one nucleotide at a time. A camera takes images of the fluorescently labeled nucleotides, then the dye along with the terminal 3′ blocker is chemically removed from the DNA, allowing the next cycle.

Helicos's single-molecule sequencing uses DNA fragments with added polyA tail adapters, which are attached to the flow cell surface. At each cycle, DNA polymerase and a single species of fluorescently labeled nucleotide are added, resulting in template-dependent extension of the surface-immobilized primer-template duplexes. The reads are performed by the Helioscope sequencer. After acquisition of images tiling the full array, chemical cleavage and release of the fluorescent label permits the subsequent cycle of extension and imaging.

Sequencing by synthesis (SBS), like the “old style” dye-termination electrophoretic sequencing, relies on incorporation of nucleotides by a DNA polymerase to determine the base sequence. A DNA library with affixed adapters is denatured into single strands and grafted to a flow cell, followed by bridge amplification to form a high-density array of spots onto a glass chip. Reversible terminator methods use reversible versions of dye-terminators, adding one nucleotide at a time, detecting fluorescence at each position by repeated removal of the blocking group to allow polymerization of another nucleotide. The signal of nucleotide incorporation can vary with fluorescently labeled nucleotides, phosphate-driven light reactions and hydrogen ion sensing having all been used. Examples of SBS platforms include Illumina GA and HiSeq 2000. The MiSeq® personal sequencing system (Illumina, Inc.) also employs sequencing by synthesis with reversible terminator chemistry.

In contrast to the sequencing by synthesis method, the sequencing by ligation method uses a DNA ligase to determine the target sequence. This sequencing method relies on enzymatic ligation of oligonucleotides that are adjacent through local complementarity on a template DNA strand. This technology employs a partition of all possible oligonucleotides of a fixed length, labeled according to the sequenced position. Oligonucleotides are annealed and ligated and the preferential ligation by DNA ligase for matching sequences results in a dinucleotide encoded color space signal at that position (through the release of a fluorescently labeled probe that corresponds to a known nucleotide at a known position along the oligo). This method is primarily used by Life Technologies' SOLiD™ sequencers. Before sequencing, the DNA is amplified by emulsion PCR. The resulting beads, each containing only copies of the same DNA molecule, are deposited on a solid planar substrate.

SMRT™ sequencing is based on the sequencing by synthesis approach. The DNA is synthesized in zero-mode wave-guides (ZMWs)-small well-like containers with the capturing tools located at the bottom of the well. The sequencing is performed with use of unmodified polymerase (attached to the ZMW bottom) and fluorescently labeled nucleotides flowing freely in the solution. The wells are constructed in a way that only the fluorescence occurring at the bottom of the well is detected. The fluorescent label is detached from the nucleotide at its incorporation into the DNA strand, leaving an unmodified DNA strand.

### Methods for Enhancing Immunogenic Response to Vaccines

In one aspect, the present disclosure provides a method for identifying a subject that will show an enhanced immunogenic response to a vaccine comprising: detecting the presence of a wild-type rs3901533 SNP (e.g., GRCh38.p12 chr12:10124484A) in at least one CLEC7A polynucleotide in a biological sample obtained from the subject, wherein the presence of the wild-type rs3901533 SNP in at least one CLEC7A polynucleotide indicates that the subject will show an enhanced immunogenic response to a vaccine. In certain embodiments, the method further comprises administering to the subject an effective amount of a vaccine and an effective amount of a yeast beta-glucan comprising a plurality of β-(1,3) side chains linked to a β-(1,3) backbone via β-(1,6) linkages, and wherein the yeast beta-glucan has a range of average molecular weights from about 6 kDa to about 30 kDa; optionally wherein the vaccine comprises at least one antigen that is linked to a carrier, and optionally wherein the antigen is a peptide, a polypeptide, a nucleic acid, a carbohydrate, a lipid, or a whole tumor cell. In some embodiments, the SNP is detected via next-generation sequencing, PCR, real-time quantitative PCR (qPCR), digital PCR (dPCR), Southern blotting, Reverse transcriptase-PCR (RT-PCR), Northern blotting, microarray, dot or slot blots, in situ hybridization, or fluorescent in situ hybridization (FISH). The biological sample may comprise genomic DNA and/or peripheral blood mononuclear cells. Additionally or alternatively, in some embodiments, the immunogenicity of the vaccine in the subject is increased compared to that observed in a control subject that does not harbor the wild-type rs3901533 SNP.

Additionally or alternatively, in some embodiments, the vaccine is a poorly immunogenic antigen-specific vaccine or a whole cell tumor vaccine. In any and all embodiments of the methods disclosed herein, the at least one antigen is associated with a disease or infection. Examples of such diseases and infections include, but are not limited to neurodegenerative disease, Alzheimer's Disease, melanoma, neuroblastoma, glioma, small cell lung cancer, t-ALL, breast cancer, brain tumors, retinoblastoma, Ewing's sarcoma, osteosarcoma, ovarian cancer, non-Hodgkin's lymphoma, Epstein-Barr related lymphoma, Hodgkin's lymphoma, leukemia, epidermoid carcinoma, prostate cancer, renal cell carcinoma, transitional cell carcinoma, lung cancer, colon cancer, liver cancer, stomach cancer, gastrointestinal cancer, pancreatic cancer, HIV, tuberculosis, malaria, influenza, Ebola, chicken pox, Hepatitis B, HPV, tetanus, pneumococcus, measles, mumps, rubella, influenza, polio, diphtheria, tetanus, pertussis, Rous Sarcoma Virus, rabies, and rotavirus.

Additionally or alternatively, in some embodiments, the structure of the at least one antigen is

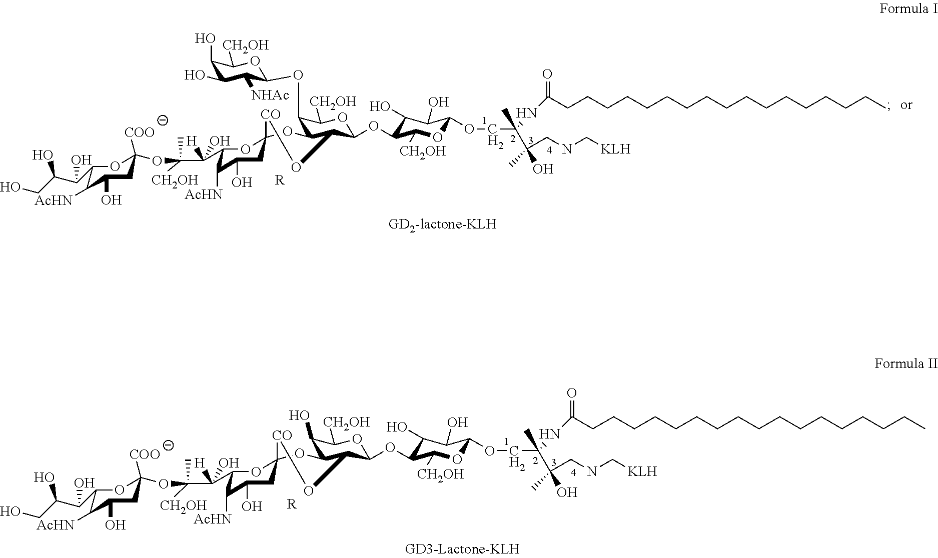

. Additionally or alternatively, in some embodiments, the at least one antigen is inactivated, partially purified or recombinant hemagglutinin (HA) protein or fucosyl GM1. Examples of the carrier include keyhole limpet hemocyanin, serum globulins, serum albumins, and ovalbumins.

Additionally or alternatively, in some embodiments, the vaccine and the yeast beta-glucan are administered separately, simultaneously or sequentially. In certain embodiments, the vaccine is administered intravenously, intramuscularly, intraarterially, intrathecally, intracapsularly, intraorbitally, intradermally, intraperitoneally, transtracheally, subcutaneously, intracerebroventricularly, orally or intranasally. In some embodiments, the yeast beta-glucan is administered intravenously, intramuscularly, intraarterially, intrathecally, intracapsularly, intraorbitally, intradermally, intraperitoneally, transtracheally, subcutaneously, intracerebroventricularly, orally or intranasally.

Additionally or alternatively, in some embodiments, administration of the vaccine and the yeast beta-glucan results in about a 1.5-fold, a 2-fold, a 2.5 fold, a 3-fold, a 3.5 fold, a 4-fold, a 4.5 fold, a 5-fold, a 5.5 fold, a 6-fold, a 6.5 fold, a 7-fold, a 7.5 fold, an 8-fold, an 8.5 fold, a 9-fold, a 9.5 fold, or 10-fold increase in therapeutic antibody titer levels (e.g., but not limited to anti-GD2 or anti-GD3) in the subject compared to that observed in the subject prior to administration of the vaccine and the yeast beta-glucan. In certain embodiments, administration of the vaccine and the yeast beta-glucan results in the persistence of therapeutic antibody titer levels (e.g., but not limited to anti-GD2 or anti-GD3) in the subject. In any of the above embodiments of the methods disclosed herein, administration of the yeast beta-glucan prolongs survival and/or prevents tumor recurrence in the subject.

In another aspect, the present disclosure provides a method for treating a metastasis-prone cancer, a neurodegenerative disease, or an infection in a subject in need thereof comprising administering to the subject an effective amount of a vaccine and an effective amount of a yeast beta-glucan comprising a plurality of β-(1,3) side chains linked to a β-(1,3) backbone via β-(1,6) linkages, wherein the yeast beta-glucan has a range of average molecular weights from about 6 kDa to about 30 kDa, and wherein the subject harbors a wild-type rs3901533 (e.g., GRCh38.p12 chr12:10124484A) SNP in at least one CLEC7A polynucleotide. In some embodiments, the vaccine comprises at least one antigen that is optionally linked to a carrier, and wherein the antigen is a peptide, a polypeptide, a nucleic acid, a carbohydrate, a lipid, or a whole tumor cell. The SNP may be detected via next-generation sequencing, PCR, real-time quantitative PCR (qPCR), digital PCR (dPCR), Southern blotting, Reverse transcriptase-PCR (RT-PCR), Northern blotting, microarray, dot or slot blots, in situ hybridization, or fluorescent in situ hybridization (FISH). Additionally or alternatively, in some embodiments, administration of the vaccine and the yeast beta-glucan protects the subject from metastasis, elevates helper T cell response to vaccines, and/or promotes progression free survival in the subject.

Examples of metastasis-prone cancers, neurodegenerative diseases, or infections include, but are not limited to Alzheimer's Disease, melanoma, neuroblastoma, glioma, small cell lung cancer, t-ALL, breast cancer, brain tumors, retinoblastoma, Ewing's sarcoma, osteosarcoma, ovarian cancer, non-Hodgkin's lymphoma, Epstein-Barr related lymphoma, Hodgkin's lymphoma, leukemia, epidermoid carcinoma, prostate cancer, renal cell carcinoma, transitional cell carcinoma, lung cancer, colon cancer, liver cancer, stomach cancer, gastrointestinal cancer, pancreatic cancer, HIV, tuberculosis, malaria, influenza, Ebola, chicken pox, Hepatitis B, HPV, tetanus, pneumococcus, measles, mumps, rubella, influenza, polio, diphtheria, tetanus, pertussis, Rous Sarcoma Virus, rabies, and rotavirus. Additionally or alternatively, in some embodiments, wherein the yeast beta-glucan is administered intravenously, intramuscularly, intraarterially, intrathecally, intracapsularly, intraorbitally, intradermally, intraperitoneally, transtracheally, subcutaneously, intracerebroventricularly, orally or intranasally.

In some embodiments of the methods disclosed herein, the yeast beta-glucan is administered one, two, three, four, or five times per day. In some embodiments, the yeast beta-glucan is administered more than five times per day. Additionally or alternatively, in some embodiments, the yeast beta-glucan is administered every day, every other day, every third day, every fourth day, every fifth day, or every sixth day. In some embodiments, the yeast beta-glucan is administered weekly, bi-weekly, tri-weekly, or monthly. In some embodiments, the yeast beta-glucan is administered for a period of one, two, three, four, or five weeks. In some embodiments, the yeast beta-glucan is administered for six weeks or more. In some embodiments, the yeast beta-glucan is administered for twelve weeks or more. In some embodiments, the yeast beta-glucan is administered for a period of less than one year. In some embodiments, the yeast beta-glucan is administered for a period of more than one year. In some embodiments, the yeast beta-glucan is administered throughout the subject's life.

In some embodiments of the methods of the present technology, the yeast beta-glucan is administered daily for 1 week or more. In some embodiments of the methods of the present technology, the yeast beta-glucan is administered daily for 2 weeks or more. In some embodiments of the methods of the present technology, the yeast beta-glucan is administered daily for 3 weeks or more. In some embodiments of the methods of the present technology, the yeast beta-glucan is administered daily for 4 weeks or more. In some embodiments of the methods of the present technology, the yeast beta-glucan is administered daily for 6 weeks or more. In some embodiments of the methods of the present technology, the yeast beta-glucan is administered daily for 12 weeks or more. In some embodiments, the yeast beta-glucan is administered daily throughout the subject's life. In certain embodiments, the yeast beta-glucan is administered daily for one or more days (1-14 days), followed by one or more days (1-14 days) of no yeast beta-glucan treatment for a total of 1, 2, 3, 4, 5, 6, 7, 8, 9, 10, 11, 12, 13, 14, or 15 or more cycles.

In any of the preceding embodiments of the methods disclosed herein, the subject is an immunocompromised subject, a pediatric subject, a geriatric subject, a relapsed subject, or a healthy subject. In certain embodiments, the subject has been exposed to chemotherapy or radiotherapy. Additionally or alternatively, in some embodiments, the subject is human. In any and all embodiments of the methods disclosed herein, the subject is homozygous (A/A) or heterozygous (C/A) for the wild-type rs3901533 SNP.

### Kits

The present disclosure also provides kits for detecting the presence of wild-type and/or mutant rs3901533 SNPs in nucleic acids encoding dectin-1. Kits of the present technology comprise one or more primer pairs and/or probes that selectively hybridize and are useful in amplifying wild-type (A/A) and/or mutant (A/C or C/C) alleles of the dectin-1 SNP rs3901533, and optionally instructions for use. In some embodiments, the kits of the present technology comprise a single primer pair and/or a single probe that hybridizes to a wild-type (A/A) or mutant (A/C or C/C) allele of the dectin-1 SNP rs3901533. In other embodiments, the kits of the present technology comprise multiple primer pairs and/or multiple probes that hybridize to wild-type (A/A) and mutant (A/C or C/C) alleles of the dectin-1 SNP rs3901533. Additionally or alternatively, in some embodiments, the kits further include probes that comprise a nucleic acid sequence of SEQ ID NO: 1, SEQ ID NO: 2, SEQ ID NO: 3, or SEQ ID NO: 4. Additionally or alternatively, in some embodiments, the kits of the present technology comprise a forward primer and a reverse primer, wherein the forward primer hybridizes toward the 5′ end of the wild-type or a mutant rs3901533 SNP and wherein the reverse primer hybridizes toward the 3′ end of the wild-type or a mutant rs3901533 SNP.

Additionally or alternatively, in some embodiments, the kits further comprise buffers, enzymes having polymerase activity, enzymes having polymerase activity and lacking 5′→3′ exonuclease activity or both 5′→3′ and 3′→5′ exonuclease activity, enzyme cofactors such as magnesium or manganese, salts, chain extension nucleotides such as deoxynucleoside triphosphates (dNTPs), modified dNTPs, nuclease-resistant dNTPs or labeled dNTPs, necessary to carry out an assay or reaction, such as amplification and/or detection of wild-type and/or mutant rs3901533 SNPs in nucleic acids encoding dectin-1. In one embodiment, the kits of the present technology further comprise a positive control nucleic acid sequence and a negative control nucleic acid sequence to ensure the integrity of the assay during experimental runs. The kit may also comprise instructions for use, software for automated analysis, containers, packages such as packaging intended for commercial sale and the like.

The kits of the present technology can also include other necessary reagents to perform any of the NGS techniques disclosed herein. For example, the kit may further comprise one or more of: adapter sequences, barcode sequences, reaction tubes, ligases, ligase buffers, wash buffers and/or reagents, hybridization buffers and/or reagents, labeling buffers and/or reagents, and detection means. The buffers and/or reagents are usually optimized for the particular amplification/detection technique for which the kit is intended. Protocols for using these buffers and reagents for performing different steps of the procedure may also be included in the kit.

The kits of the present technology may include components that are used to prepare nucleic acids from a biological sample for the subsequent amplification and/or detection of wild-type and/or mutant rs3901533 SNPs in nucleic acids encoding dectin-1. Such sample preparation components can be used to produce nucleic acid extracts from tissue samples. The test samples used in the above-described methods will vary based on factors such as the assay format, nature of the detection method, and the specific tissues, cells or extracts used as the test sample to be assayed. Methods of extracting nucleic acids from samples are well known in the art and can be readily adapted to obtain a sample that is compatible with the system utilized. Automated sample preparation systems for extracting nucleic acids from a test sample are commercially available, e.g., Roche Molecular Systems' COBAS AmpliPrep System, Qiagen's BioRobot 9600, and Applied Biosystems' PRISM™ 6700 sample preparation system.

Additionally or alternatively, in some embodiments, the kits of the present technology further comprise a solubilized yeast beta-glucan, a vaccine, and instructions for use, wherein the solubilized yeast beta-glucan comprises a plurality of β-(1,3) side chains linked to a β-(1,3) backbone via β-(1,6) linkages, and has a range of average molecular weights from about 6 kDa to about 30 kDa. In some embodiments of the kits of the present technology, the vaccine comprises at least one antigen that is optionally linked to a carrier, wherein the at least one antigen is a peptide, a polypeptide, a nucleic acid, a carbohydrate, or a lipid. The at least one antigen may be a peptide, a polypeptide, a nucleic acid, a carbohydrate, or a lipid that is associated with any disease or infection, including but not limited to those disclosed herein.

Additionally or alternatively, in some embodiments of the kits of the present technology, the at least one antigen is one or more of GD2 lactone, GD3 lactone, fucosyl GM1, and hemagglutinin (HA) protein (e.g., inactivated, partially purified or recombinant hemagglutinin). Examples of the carrier include keyhole limpet hemocyanin, serum globulins, serum albumins, and ovalbumins.

Additionally or alternatively, in some embodiments of the kits, the solubilized yeast beta-glucan and/or the vaccine is formulated for intravenous, intramuscular, intraarterial, intrathecal, intracapsular, intraorbital, intradermal, intraperitoneal, transtracheal, subcutaneous, intracerebroventricular, oral or intranasal administration.

Optionally, the above described components of the kits of the present technology are packed in suitable containers and labeled for enhancing the immunogenicity of a vaccine in a subject. The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, etc. The kits may optionally include instructions customarily included in commercial packages of therapeutic products, that contain information about, for example, the indications, usage, dosage, manufacture, administration, contraindications and/or warnings concerning the use of such therapeutic products.

The kits may also include additional agents that are useful for detecting the therapeutic antibody titer levels in a biological sample including, but not limited to, e.g., serum, plasma, lymph, cystic fluid, urine, stool, cerebrospinal fluid, ascitic fluid or blood and including biopsy samples of body tissue. For example, the kit may comprise: one or more antigens (e.g., but not limited to GD2 or GD3) capable of binding to the induced antibodies present in the biological sample, a means for determining the amount of the induced antibodies present in the biological sample, and a means for comparing the amount of the immunoreactive induced antibodies in the biological sample with a standard. The one or more antigens may be labeled. The kit components, (e.g., reagents) can be packaged in a suitable container. The kit can further comprise instructions for using the kit to detect the immunoreactive induced antibodies.

The kit can also comprise, e.g., a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit, e.g., for detection of induced antibodies in vitro or in vivo, or for enhancing the immunogenicity of a vaccine in a subject in need thereof. In certain embodiments, the use of the reagents can be according to the methods of the present technology.

### EXAMPLES

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### Example 1: Experimental Materials and Methods

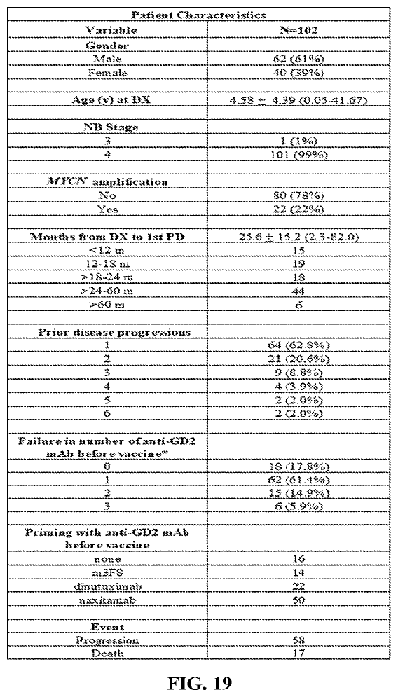

Patients. The first cohort of patients treated with vaccine consists of one hundred and two high risk neuroblastoma (HR-NB) patients with prior disease progressions (see FIG. 19; Clinicaltrials.gov NCT00911560). Eligibility criteria for this Phase II trial included patients with MYCN-amplified stage 2/3/4/4S at any age or MYCN-non-amplified metastatic disease diagnosed at ≥18 months of age; absolute lymphocyte and neutrophil counts each at ≥500/μl. In accordance with the Common Terminology Criteria for Adverse Events, Version 3.0 (CTCAEv3.0), major organ toxicity was ≤grade 3, and neurologic status was ≤grade 1.

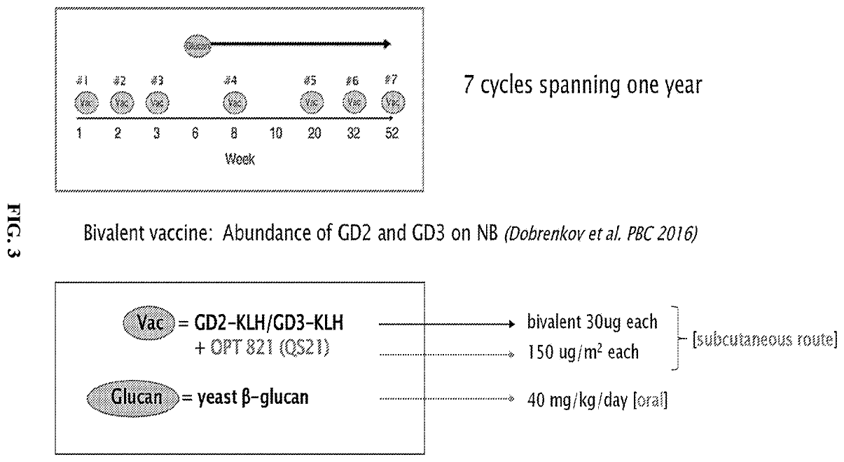

Patients had all achieved complete remission after salvage therapy before entering this Phase II trial. All prior therapies had to be completed ≥21 days before enrollment. None had received any prior GD2/GD3 vaccine. Patients received 7 subcutaneous vaccine injections (week 1-2-3-8-20-32-52) plus oral μ-glucan starting week 6 at 40 mg/kg/day, 14 days on/14 days off, up to one cycle after the last vaccination. See FIG. 3. Each vaccine injection consisted of 30 μg each of GD2 and GD3, lactonized and conjugated to the immunogenic carrier protein keyhole limpet hemocyanin (KLH) and mixed with the saponin OPT-821 adjuvant (150 μg/m²). The treatment schedule and the source of vaccine and β-glucan were identical to the Phase I trial (Kushner et al., Clin Cancer Res 20:1375-82 (2014)). Once patients progressed on vaccine, they no longer received any additional injections.

Blood collection. Patients had blood draws performed before each vaccine injection, as well as shipment of their specimen after cycle 3 and cycle 6 to the laboratory. Sera were collected and frozen at −20° C. for batch analyses.

### Quantitation of Serum Anti-Vaccine Antibody by ELISA.

Anti-GD2-IgG1: Purified GD2 at 20 ng per well was coated on CDGH rows of 96-well microtiter plates and air-dried overnight. Blocking was performed using 200 μl/well of 0.5% bovine serum albumin (BSA) in 1× phosphate-buffered solution (PBS) for one hour at room temperature, and then washed with 1×PBS. Patient serum (1:30 dilution in 0.5% BSA) was added to the designated wells with and without GD2 at 50 μl/well in duplicate. Anti-GD2 antibody Hu3F8-IgG1 was used to generate a standard curve. Both standards and samples were incubated for 2.5 hours at 37° C. After washing with PBS, peroxidase-conjugated mouse anti-human IgG1 at 1:1000 dilution was added at 100 μl/well. Upon incubation at 4° C. for 1 hour and washing with PBS, color reaction with chromogen o-phenylenediamine and substrate hydrogen peroxide was added to the plates for 30 minutes at room temperature in the dark. The reaction was stopped using sulfuric acid, and the optical density was read using ELISA plate reader at 490 nm. Patient sera were quantified in ng/ml based on the standard curve.

Anti-GD2-IgM: The anti-GD2-IgM protocol was similar to the anti-GD2-IgG1 assay described above, with the exception of using a high IgM titer human serum as the standard curve, plus the addition of peroxidase-conjugated mouse anti-human IgM as the secondary antibody. Patient sera were quantified in Units/ml based on the standard curve.

Anti-GD3-IgG1: Purified GD3 at 20 ng per well was coated on CDGH rows of 96-well microtiter plates and air-dried overnight. ELISA was similar to the anti-GD2-IgG1 assay described above with the exception of using chimeric IgG1 anti-GD2 K6G as the standard.

Anti-KLH-IgG1: Keyhole limpet hemocyanin was dissolved in PBS and coated at 250 ng per well onto CDGH rows of 96 well microtiter plates for one hour at 37° C. Upon aspiration, plates were stored at −20° C. till the day of the experiment. The standard was a high titer human serum, and patient sera were expressed in U/ml of IgG1.

Minimal residual disease (MRD) detection. Quantitative reverse transcription-polymerase chain reaction (qRT-PCR) was performed as previously described in Cheung et al., J Clin Oncol 30:3264-70 (2012) to assess MRD in heparinized BM aspirates pooled from four sites. The MRD marker panel included cyclin D1 (CCND1), GD2 synthase (B4GALNT1), ISL LIM homeobox 1 (ISL1) and paired-like homeobox 2b (PHOX2B). β2 microglobulin (β2M) was used as the endogenous control, and NB cell line NMB7 as the positive control. Each sample was quantified using the comparative CT method as fold-difference relative to NMB7.

Dectin-1 (CLEC7A) polymorphism genotyping. 40 ng of genomic DNA was used for allelic discrimination PCR using Applied Biosystems Sequence detection system 7300. Applied Biosystems TaqMan SNP Genotyping Assays use TaqMan 5′-nuclease chemistry to amplify and detect specific polymorphisms in purified genomic DNA samples. Each assay enables genotyping of individuals for a single nucleotide polymorphism (SNP) and consists of two sequence-specific primers and two TaqMan minor groove binder (MGB) probes with non-fluorescent quenchers (NFQ). One probe is labeled with VIC dye to detect the Allele 1 sequence; the second probe is labeled with FAM dye to detect the Allele 2 sequence. SNP genotyping included rs16910526 (A/C), rs7309123(C/G), rs3901533 (A/C), and rs16910527 (A/C).

Probes include:

rs3901533 SNP ID: C_7433799_40
Context Sequence [VIC/FAM]:
(SEQ ID NO: 3)
CGGGGATCTCTTTAAACTCCTATAG[A]ATTTATTGCTAATGCCAGATAT
TTA
(SEQ ID NO: 4)
CGGGGATCTCTTTAAACTCCTATAG[C]ATTTATTGCTAATGCCAGATAT
TTA
rs7309123 SNP ID: C_3130832_10 Gene= CLEC7A Chr.12
Context Sequence [VIC/FAM]:
(SEQ ID NO: 5)
GTATACGTGTTGAAATAATAGATTT[C]AGAAAGAACTAAACTAAAATTA
TAA
(SEQ ID NO: 6)
GTATACGTGTTGAAATAATAGATTT[G]AGAAAGAACTAAACTAAAATTA
TAA
C/G, Transversion Substitution
rs16910526 SNP ID: C_33748481_10 Gene = CLEC7A
Chr.12
Context Sequence [VIC/FAM]:
(SEQ ID NO: 7)
TTGAAAACTTCTTCTCACAAATACT[A]TATGAGGGCACACTACACAGTT
GGT
(SEQ ID NO: 8)
TTGAAAACTTCTTCTCACAAATACT[C]TATGAGGGCACACTACACAGTT
GGT
A/C, Transversion Substitution
rs16910527 SNP ID: C_33748482_10 Gene = CLEC7A
Chr.12
Context Sequence [VIC/FAM]:
(SEQ ID NO: 9)
TTGGTCATAAATGACTGACACGTGA[A]TCCATACACAATTTGGAGATGG
GTT
(SEQ ID NO: 10)
TTGGTCATAAATGACTGACACGTGA[C]TCCATACACAATTTGGAGATGG
GTT

PCR conditions included an initial holding step of 10 minutes at 95° C., and 40 cycles of 15 second denaturation at 92° C., and annealing/extension for 1 minute at 60° C. Allelic discrimination of dectin-1 as wild-type, heterozygote, and mutant was identified by the ABI Sequence Detection Systems software.

Extent of disease evaluation. Computed tomography (CT) and ¹²³I-metaiodobenzylguanidine (MIBG) scintigraphy were performed at enrollment and then every 10-12 weeks through 24 months. Bone marrow (BM) histology (aspirates and biopsies from bilateral posterior and anterior iliac crests) was assessed at enrollment, at least once <6 months from enrollment, and after the final (#7) vaccine injection. Using the International NB Response Criteria, expanded to include 123I-MIBG findings, complete remission (CR) was defined as absence of active NB by all studies, and progressive disease (PD) was defined as a new lesion or >25% increase in any disease marker.

Biostatistics. Survival was estimated from the first dose of vaccine through PD or death for progression-free survival (PFS), and through death for overall survival (OS). Alive patients as well as patients alive without progression for PFS were censored at the date of their last follow-up. Kaplan-Meier estimates and landmark Cox Regression models were used for survival estimates and prognostic impact analyses. Week 8 from the start of vaccine treatment was used as landmark time to study the prognostic impact of having antibody titer level by week 8 along with other prognostic factors. ϰ2 was used to categorize antibody titers (using binary median titer cut-off) with SNP genotyping results.

### Example 2: Effects of Anti-GD2/Anti-GD3 Bivalent Vaccine and β-Glucan Therapy in Phase II Neuroblastoma Trial

CR2 Cohort: Patients with High Risk Neuroblastoma with Previous History of Disease Progression. In this Phase II trial (Clinicaltrials.gov NCT00911560), 102 HR-NB patients who achieved complete remission after salvage therapy were enrolled (termed ≥2^{nd} remission, CR2). Patients received 7 subcutaneous vaccine injections (week 1-2-3-8-20-32-52) plus oral β-glucan (starting in week 6 at 40 mg/kg/day, 14 days on/14 days off). See FIG. 3. Each subcutaneous vaccine injection consisted of 30 μg each of GD2/GD3 vaccine and mixed with the saponin OPT-821 adjuvant. Serum anti-vaccine antibody titers were quantified by ELISA. Kaplan-Meier statistics and landmark Cox Regression models were used for survival estimates and prognostic impact analyses.

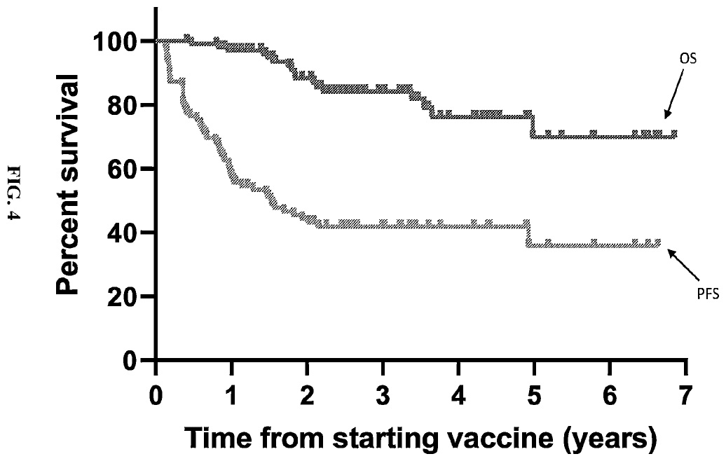

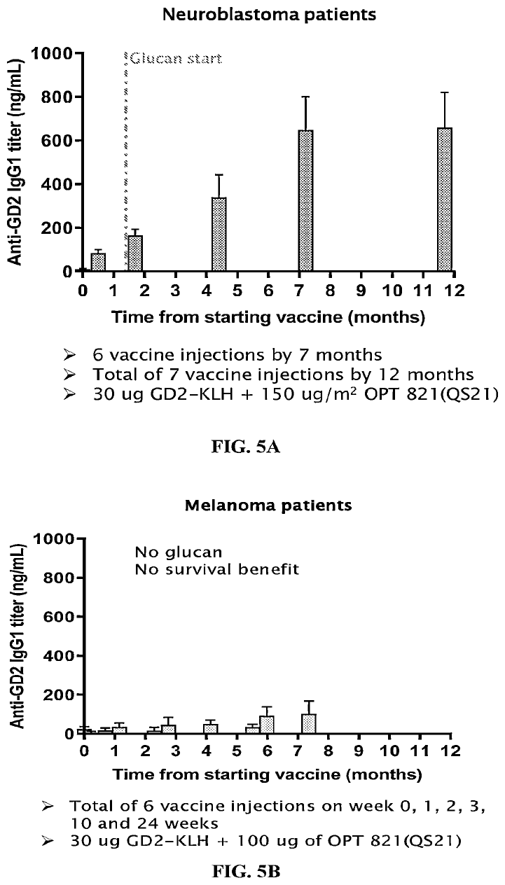

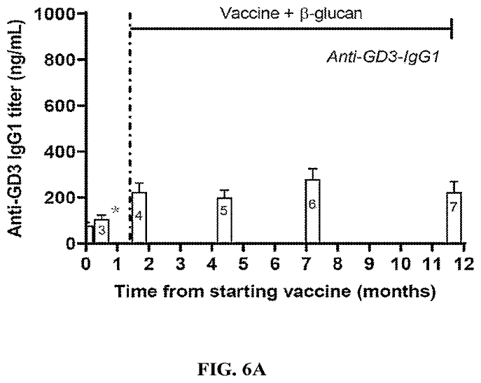

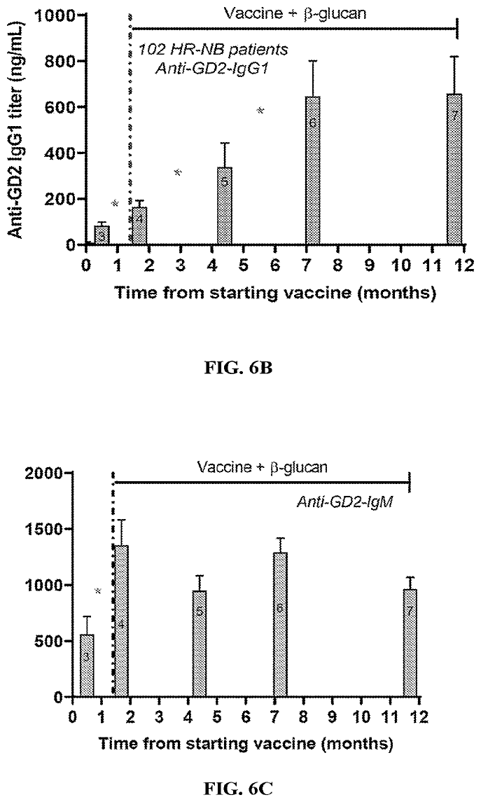

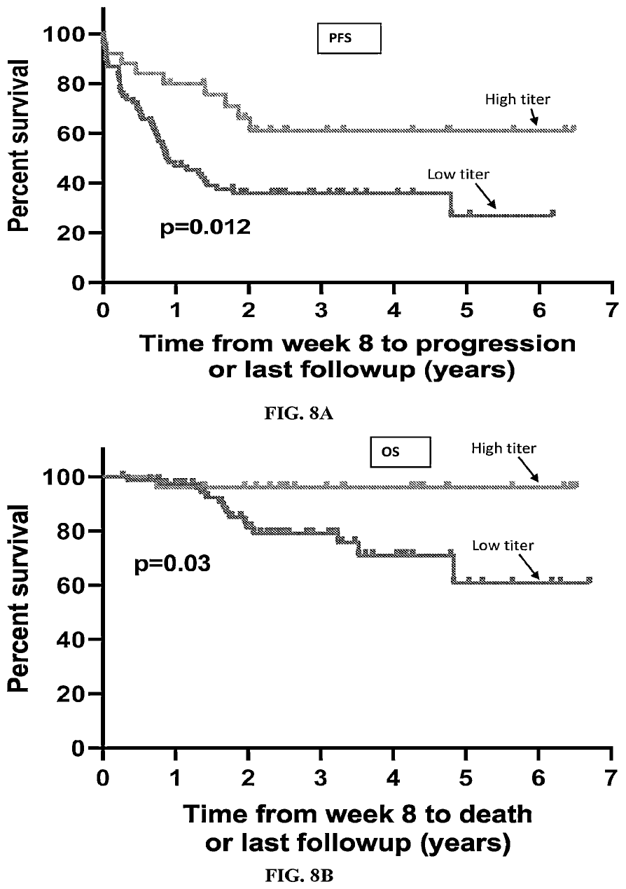

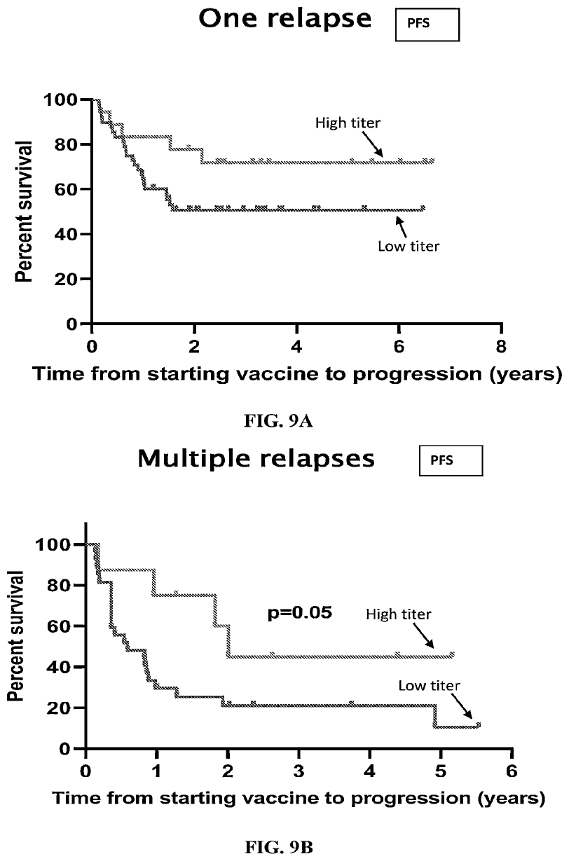

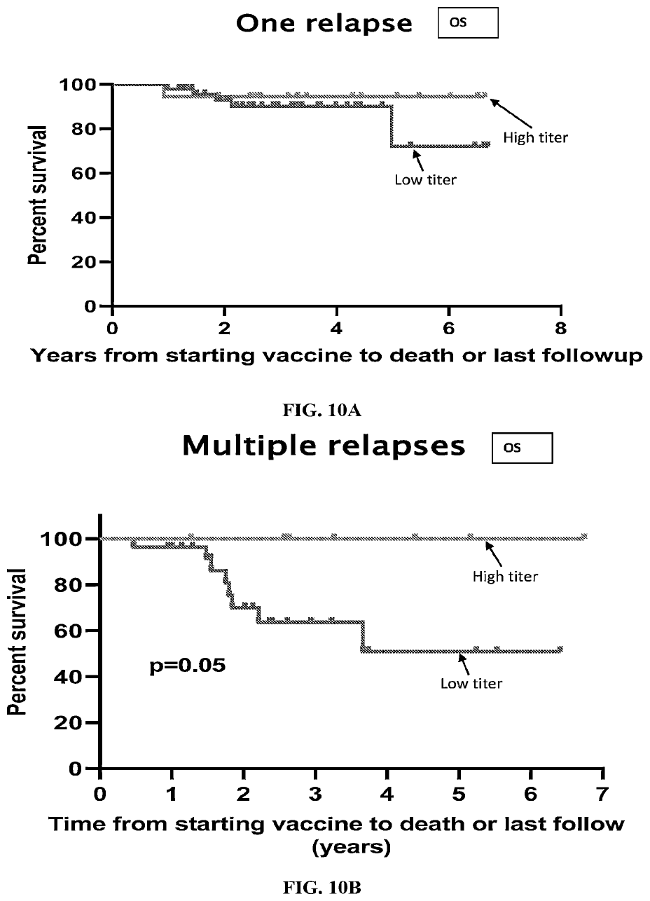

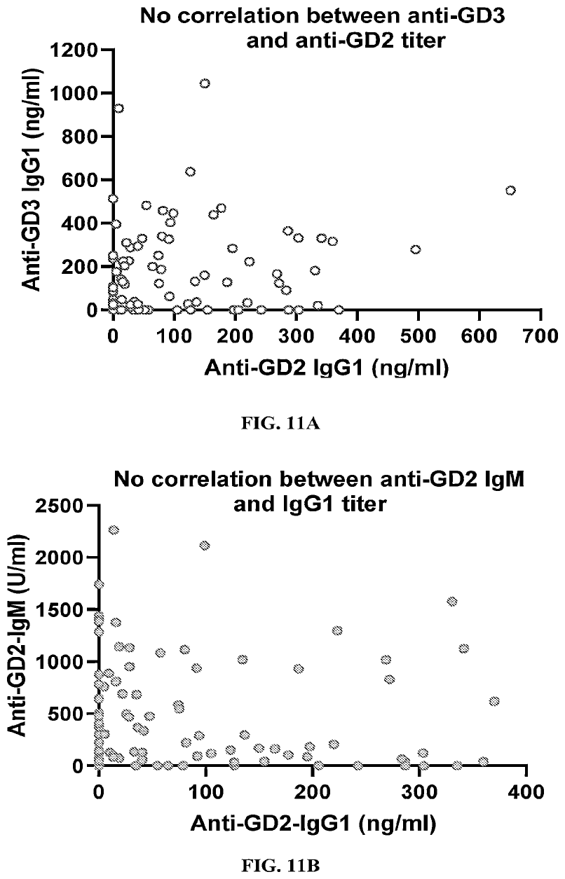

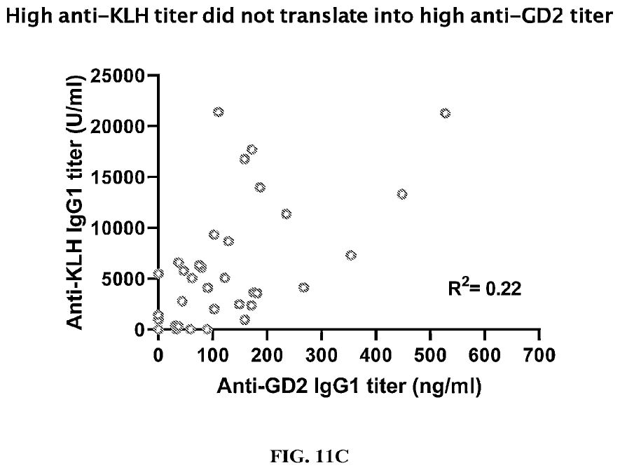

Among 102 patients, 63% had one, 21% had 2, and 17% had 3-6 prior disease progressions. See FIG. 19. 83/101 patients had failed prior anti-GD2 mAb (m3F8, dinutuximab, or naxitamab) therapy before the anti-GD2/anti-GD3 bivalent vaccine was administered: one mAb (n=62), two mAbs (n=15), or all three (n=6). Common toxicities were self-limited injection-related local reactions and fever. No pain, neuropathy, or grade 3/4 toxicities occurred during or post treatment. As shown in FIG. 4, progression-free survival (PFS) was 44%±5% and overall survival (OS) was 88%±4% at 2 years, and 36%±7% and 70%±8% at 5 years, respectively. Of 102 patients, only 101 had available sera for seroconversion analysis. As shown in FIGS. 5A-5B and FIGS. 6A-6C, serum anti-GD2 (IgG1 and IgM) and anti-GD3 (IgG1) titers had marked increases following the initiation of β-1,3/1,6-glucan at week 6. In univariate analyses, favorable prognostic factors included: one versus ≥2 prior disease progressions (PFS p=0.005, OS p=0.04), none versus any prior anti-GD2 mAb failures (PFS p=0.004, OS p=0.01); and the induction of ≥150 ng/ml anti-GD2-IgG1 titers by week 8 (PFS p=0.02, OS p=0.06) (FIGS. 8A-8B). Factors not prognostic included: time to first NB progression, MYCN amplification status, anti-GD2-IgM (FIG. 11B), anti-GD3-IgG1 (FIG. 11A), or anti-KLH-IgG1 (FIG. 11C) titers, and priming with anti-GD2 mAb right before vaccine. In multivariate analyses, week 8 anti-GD2-IgG1 titer ≥150 ng/ml yielded a hazard ratio (HR) of 0.41 [0.20, 0.83], p=0.01 for PFS, and HR=0.15 [0.02, 1.12], p=0.06 for OS. The second independent prognostic variable was the number of prior disease progressions (≥2 vs 1), yielding HR of 2.12 [1.26, 3.58], p=0.005 for PFS, and HR=2.77 (1.03, 7.47), p=0.04 for OS. See FIGS. 9A-9B and FIGS. 10A-10B.

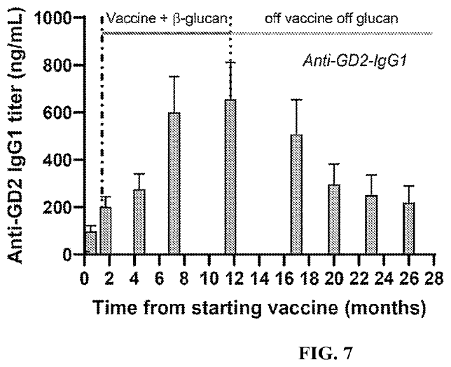

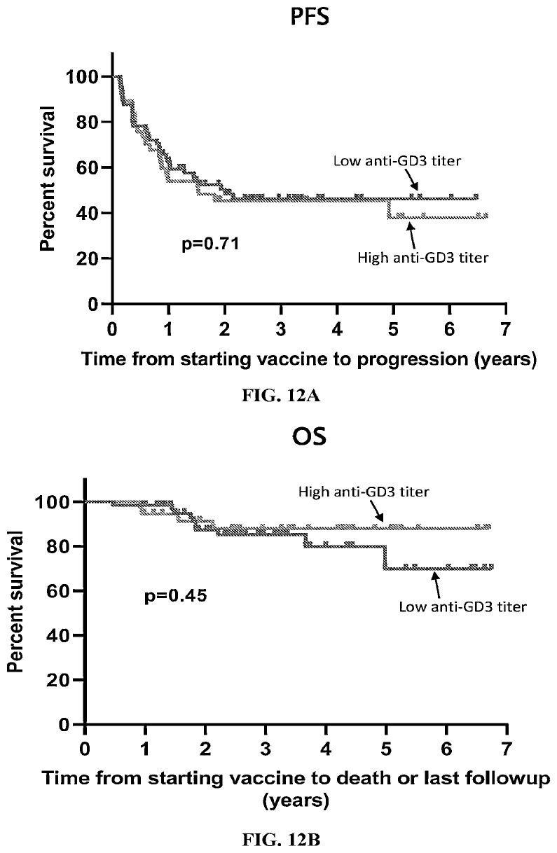

Even with prior disease progressions, anti-GD2 (though not anti-GD3; see FIGS. 12A-12B) seroconversion was associated with notable long-term survival among HR-NB patients previously thought to be unsalvageable. As shown in FIG. 7, anti-vaccine titers can persist after treatment cycle 7, though trending down over 24 months. Low levels (<1 μg/ml) of anti-GD2 or anti-GD3 antibody (similar to autoantibodies) were safe, even when persistent over months to years. Since ˜50% disease progression was between 12-24 months, these results suggest that periodic vaccine boost through 24 months to maintain high anti-GD2 IgG1 titer might prevent relapse.

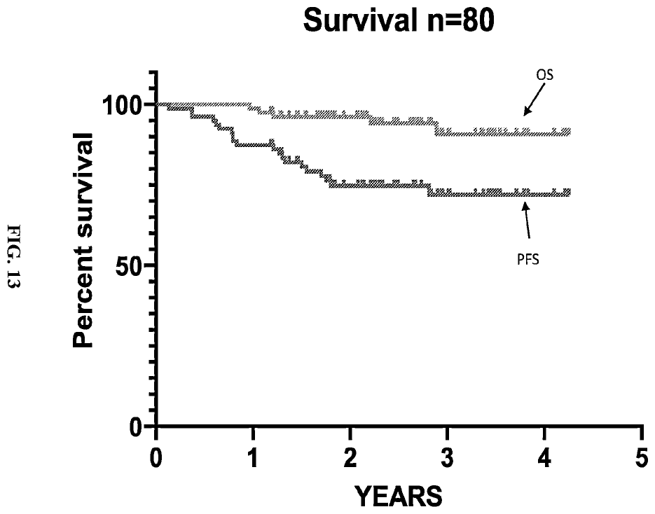

CR1 cohort: Patients with high risk neuroblastoma with no prior history of disease progression. FIG. 13 shows the high probability of PFS and OS among 80 high risk neuroblastoma patients without prior disease progression (CR1) and treated with the vaccine and β-1,3/1,6-glucan adjuvant.

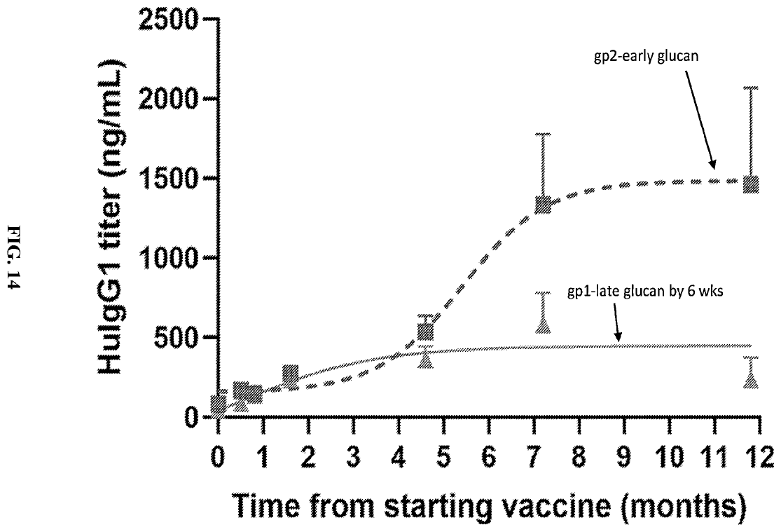

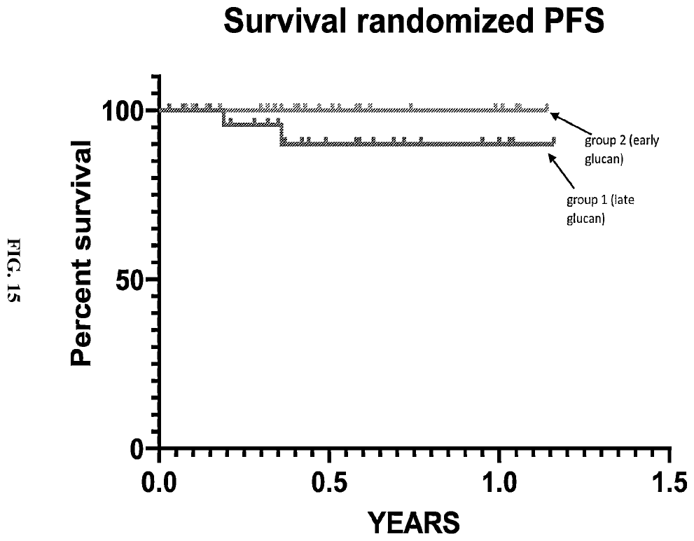

FIGS. 14-15 demonstrate that patients that received β-glucan upfront (early) induced substantially higher anti-GD2 IgG1 antibody titer and higher PFS compared to those starting glucan at week 6 (late) without any added toxicities.

### Example 3: Correlation Between Wild-type Dectin-1 SNP rs3901533 and Seroconversion

The correlation between the dectin-1 (CLEC7A) SNP rs3901533 with antibody response titer levels in the present phase II ganglioside vaccine trial was evaluated. In published studies, patients carrying the mutant allele have a diminished response to fungal infections leading to poorer survival (Zhou et al., Biosci Rep 39(11) (2019)).

Sera collected before each vaccine injection were frozen at −20° C. for batch analyses. Anti-GD2 IgG1 and IgM, as well as anti-GD3 IgG1 titers were quantified by ELISA. Genomic DNA extracted from patients' peripheral blood mononuclear cells was used to perform rs3901533 (A/C) SNP genotyping by PCR to identify wildtype (A/A), heterozygous (A/C), and mutant (C/C). Antibody titer cut points were set at median titer. Statistics were performed using chi-square. Table 2 shows the correlation between median antibody titer of anti-GD2-IgG1, anti-GD3-IgG1, anti-GD2-IgM, and anti-KLH-IgG1 among CR2 patients with dectin-1 (CLEC7A) polymorphism SNP rs3901533.

TABLE 2

| | Anti-GD2-IgG1 | | Anti-GD3-IgG1 | |
|---|---|---|---|---|
| Polymorphic | <300 | >300 | <260 | >260 |
| alleles | (ng/mL) | (ng/mL) | (ng/mL) | (ng/mL) |
| A/A | 4 (31%) | 9 (69%) | 4 (31%) | 9 (69%) |
| A/C | 30 (79%) | 8 (21%) | 27 (71%) | 11 (29%) |
| C/C | 33 (66%) | 17 (34%) | 37 (74%) | 13 (26%) |
| | p = 0.007 | | p = 0.015 | |
| | Anti-GD2-IgM | | Anti-KLH-IgG1 | |
| Polymorphic | <960 | >960 | <6400 | >6400 |
| alleles | (U/mL) | (U/mL) | (U/mL) | (U/mL) |
| A/A | 6 (46%) | 7 (54%) | 4 (31%) | 9 (69%) |
| A/C | 22 (58%) | 16 (42%) | 16 (42%) | 22 (58%) |
| C/C | 26 (52%) | 24 (48%) | 31 (62%) | 19 (38%) |
| | p = 0.70 | | p = 0.058 | |

Both anti-GD2 and anti-GD3 IgG1 antibody responses correlated with SNP rs3901533 (A/A) of dectin-1 (CLEC7A) among these 101 CR2 patients. Patients carrying the mutant allele (C/C or A/C) had significantly lower titers. The anti-GD2 IgM response, which is T helper cell independent, had no correlation with SNP rs3901533. Since neither GD2-L or GD3-L (being carbohydrate antigens) could activate helper T cells by themselves, this helper effect had to be mediated through KLH, the carrier protein. Of interest was the anti-KLH IgG1 response, which was also insignificant (p=0.13), for a protein known to stimulate a powerful T cell helper response, with probably less dependence on the adjuvants.

When the other two dectin-1 SNPs (rs16910526, rs7309123) were studied, neither showed any significant correlation with seroconversion (Table 3). Allelic frequency of homozygous mutant (G/G) for rs7309123 was 28%, whereas the allelic frequency of homozygous mutant (C/C) for rs16910526 was 8%.

TABLE 3

Anti-GD2-IgG1

| Polymorphic alleles | <300 (ng/mL) | >300 (ng/mL) |
|---|---|---|
| rs7309123 | | |
| C/C | 21 (68%) | 10 (32%) |
| C/G | 34 (71%) | 14 (29%) |
| G/G | 12 (55%) | 10 (45%) |
| | p = 0.41 | |
| rs16910526 | | |
| A/A | 49 (67%) | 24 (33%) |
| A/C | 11 (69%) | 5 (31%) |
| C/C | 7 (58%) | 5 (42%) |
| | p = 0.84 | |

To validate the correlation of dectin-1 SNP rs3901533 with antibody titer, a subsequent cohort of 77 CR1 patients with no prior history of disease progression (Clinicaltrials.gov NCT00911560), i.e. treated with vaccine in first remission (CR1), was analyzed. These patients received the same treatment as those treated in CR2. As shown in Table 4, there was a strong correlation of anti-GD2 IgG1, but not IgM seroconversion with the mutant allele.

TABLE 4
77 CR1 patients with no prior disease progressions

| | Anti-GD2-IgG1 | | Anti-GD2-IgM | |
|---|---|---|---|---|
| Polymorphic | 360 | >360 | <560 | >560 |
| alleles | (ng/mL) | (ng/mL) | (U/mL) | (U/mL) |
| A/A | 4 (36%) | 7 (64%) | 3 (27%) | 8 (73%) |
| A/C | 20 (62%) | 12 (38%) | 18 (56%) | 14 (44%) |
| C/C | 26 (76%) | 8 (24%) | 18 (53%) | 16 (47%) |
| | p = 0.05 | | p = 0.25 | |

This correlation was further validated in a third cohort of 76 patients randomized to either receiving β-glucan upfront or on the standard schedule in week 6 after the third vaccine injection. Again, Table 5 demonstrates that there was a significant correlation of seroconversion with dectin-1 SNP.

TABLE 5
76 patients in randomized vaccine trial

Anti-GD2-IgG1

| Polymorphic alleles | <220 (ng/mL) | >220 (ng/mL) |
|---|---|---|
| A/A | 3 (33%) | 6 (67%) |
| A/C | 9 (31%) | 20 (69%) |
| C/C | 23 (61%) | 15 (39%) |
| | p = 0.045 | |

Based on the learning set of 101 CR2 patients and two subsequent test sets of 71 (CR1) and 76 (randomized) patients each, genotyping of rs3901533 revealed a statistically significant association of homozygous mutant alleles C/C with lower post glucan IgG1 antibody titer for both T helper reliant gangliosides GD2 and GD3. For T cell independent IgM antibody, there was no association with dectin-1 SNP. Nor was there any significant correlation between anti-KLH antibody titers with rs3901533. These results demonstrate that β-glucan can greatly enhance the T cell dependent IgG antibody response to the vaccine, through the dectin-1 receptor mechanism, and that the rs3901533 SNP serves as a reliable and accurate biomarker for antibody response to vaccines when β-glucan is used as an adjuvant. ˜25% patients remained seronegative after 1 year of vaccine therapy, most of whom were predictable by their dectin-1 SNP rs3901533. Only a subset of the seronegative patient without the mutant allele for SNP could become seropositive by retreatment with another year of vaccine.

Accordingly, the wild-type rs3901533 (A/A) dectin-1 SNP is useful in methods for predicting whether a subject will show an enhanced immunogenic response to a vaccine.

### Equivalents

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for identifying a subject that will show an enhanced immunogenic response to a vaccine comprising:
detecting the presence of a wild-type rs3901533 SNP (e.g., GRCh38.p12 chr12:10124484A) in at least one CLEC7A polynucleotide in a biological sample obtained from the subject, wherein the presence of the wild-type rs3901533 SNP in at least one CLEC7A polynucleotide indicates that the subject will show an enhanced immunogenic response to a vaccine.

2. The method of claim 1, further comprising administering to the subject an effective amount of a vaccine and an effective amount of a yeast beta-glucan comprising a plurality of β-(1,3) side chains linked to a β-(1,3) backbone via β-(1,6) linkages, and wherein the yeast beta-glucan has a range of average molecular weights from about 6 kDa to about 30 kDa;
optionally wherein the vaccine comprises at least one antigen that is linked to a carrier, and optionally wherein the antigen is a peptide, a polypeptide, a nucleic acid, a carbohydrate, a lipid, or a whole tumor cell.

3. The method of claim 1, wherein the SNP is detected via next-generation sequencing, PCR, real-time quantitative PCR (qPCR), digital PCR (dPCR), Southern blotting, Reverse transcriptase-PCR (RT-PCR), Northern blotting, microarray, dot or slot blots, in situ hybridization, or fluorescent in situ hybridization (FISH) and/or wherein the biological sample comprises genomic DNA and/or peripheral blood mononuclear cells.

4. (canceled)

5. The method of claim 2, wherein the immunogenicity of the vaccine in the subject is increased compared to that observed in a control subject that does not harbor the wild-type rs3901533 SNP.

6. The method of claim 1, wherein the vaccine is a poorly immunogenic antigen-specific vaccine or a whole cell tumor vaccine.

7. The method of claim 2, wherein the at least one antigen is associated with a disease or infection.

8. The method of claim 7, wherein the disease or infection is selected from the group consisting of neurodegenerative disease, Alzheimer's Disease, melanoma, neuroblastoma, glioma, small cell lung cancer, t-ALL, breast cancer, brain tumors, retinoblastoma, Ewing's sarcoma, osteosarcoma, ovarian cancer, non-Hodgkin's lymphoma, Epstein-Barr related lymphoma, Hodgkin's lymphoma, leukemia, epidermoid carcinoma, prostate cancer, renal cell carcinoma, transitional cell carcinoma, lung cancer, colon cancer, liver cancer, stomach cancer, gastrointestinal cancer, pancreatic cancer, HIV, tuberculosis, malaria, influenza, Ebola, chicken pox, Hepatitis B, HPV, tetanus, pneumococcus, measles, mumps, rubella, influenza, polio, diphtheria, tetanus, pertussis, Rous Sarcoma Virus, rabies, and rotavirus.

9. The method of claim 2, wherein the structure of the at least one antigen is

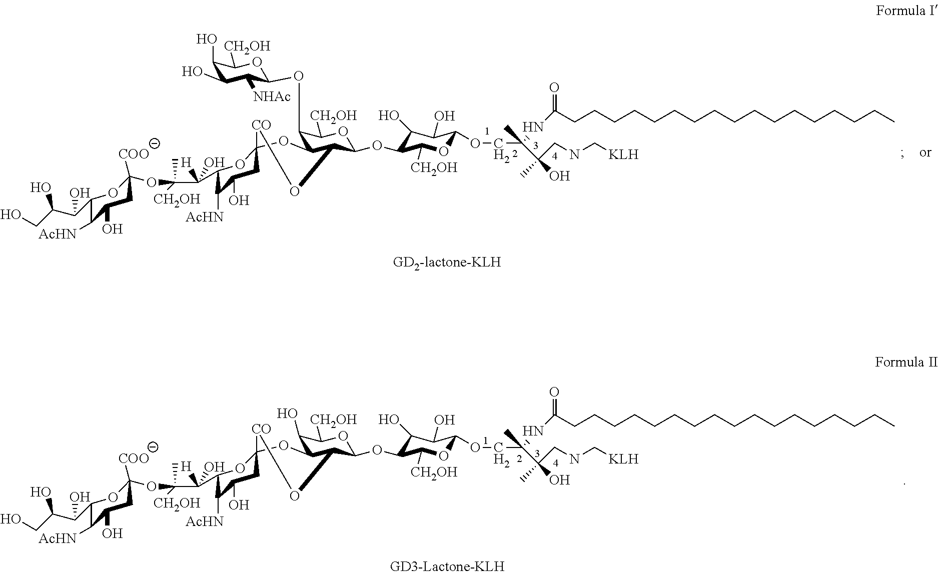

10. The method of claim 2, wherein the at least antigen is inactivated, partially purified or recombinant hemagglutinin (HA) protein or fucosyl GM1.

11. The method of claim 2, wherein the carrier is keyhole limpet hemocyanin (KLH).

12. The method of claim 2, wherein the vaccine and the yeast beta-glucan are administered separately, sequentially or simultaneously.

13. The method of claim 2, wherein the vaccine or the yeast beta-glucan is administered intravenously, intramuscularly, intraarterially, intrathecally, intracapsularly, intraorbitally, intradermally, intraperitoneally, transtracheally, subcutaneously, intracerebroventricularly, orally or intranasally.

14. (canceled)

15. The method of claim 2, wherein administration of the vaccine and the yeast beta-glucan
results in a 10-fold increase in therapeutic antibody titer levels in the subject compared to that observed in the subject prior to administration of the vaccine and the yeast beta-glucan; or
results in the persistence of therapeutic antibody titer levels in the subject.

16. The method of claim 1, wherein the subject has been exposed to chemotherapy or radiotherapy; or
wherein the subject is an immunocompromised subject, a pediatric subject, a geriatric subject, a relapsed subject, or a healthy subject or
wherein the subject is human; or
wherein the subject is homozygous or heterozygous for the wild-type rs3901533 SNP.

17. (canceled)

18. (canceled)

19. A method for treating a metastasis-prone cancer, a neurodegenerative disease, or an infection in a subject in need thereof comprising
administering to the subject an effective amount of a vaccine and an effective amount of a yeast beta-glucan comprising a plurality of β-(1,3) side chains linked to a β-(1,3) backbone via β-(1,6) linkages, wherein the yeast beta-glucan has a range of average molecular weights from about 6 kDa to about 30 kDa, and wherein the subject harbors a wild-type rs3901533 (e.g., GRCh38.p12 chr12:10124484A) SNP in at least one CLEC7A polynucleotide.

20. The method of claim 19, wherein the vaccine comprises at least one antigen that is optionally linked to a carrier, and wherein the antigen is a peptide, a polypeptide, a nucleic acid, a carbohydrate, a lipid, or a whole tumor cell.

21. The method of claim 19, wherein the SNP is detected via next-generation sequencing, PCR, real-time quantitative PCR (qPCR), digital PCR (dPCR), Southern blotting, Reverse transcriptase-PCR (RT-PCR), Northern blotting, microarray, dot or slot blots, in situ hybridization, or fluorescent in situ hybridization (FISH).

22. The method of claim 2, wherein administration of the vaccine and the yeast beta-glucan protects the subject from metastasis, elevates helper T cell response to vaccines, and/or promotes progression free survival in the subject.

23. (canceled)

24. (canceled)

25. The method of claim 1, wherein the SNP is detected using one or more detectably labelled probes comprising a nucleic acid sequence of SEQ ID NO: 1, SEQ ID NO: 2, SEQ ID NO: 3, or SEQ ID NO: 4.

26. The method of claim 1, wherein the subject is an immunocompromised subject, a pediatric subject, a geriatric subject, a relapsed subject, or a healthy subject.


SEQUENCE LISTING (10 sequences)

SEQ ID NO: 1 - DNA, 51 bp, Unknown
  Description of Unknown SNP detection sequence
ggggatctctttaaactcctatagaatttattgctaatgccagatatttaa

SEQ ID NO: 2 - DNA, 51 bp, Unknown
  Description of Unknown SNP detection sequence
ggggatctctttaaactcctatagcatttattgctaatgccagatatttaa

SEQ ID NO: 3 - DNA, 51 bp, Unknown
  Description of Unknown SNP detection sequence
cggggatctctttaaactcctatagaatttattgctaatgccagatattta

SEQ ID NO: 4 - DNA, 51 bp, Unknown
  Description of Unknown SNP detection sequence
cggggatctctttaaactcctatagcatttattgctaatgccagatattta

SEQ ID NO: 5 - DNA, 51 bp, Unknown
  Description of Unknown SNP detection sequence
gtatacgtgttgaaataatagatttcagaaagaactaaactaaaattataa

SEQ ID NO: 6 - DNA, 51 bp, Unknown
  Description of Unknown SNP detection sequence
gtatacgtgttgaaataatagatttgagaaagaactaaactaaaattataa

SEQ ID NO: 7 - DNA, 51 bp, Unknown
  Description of Unknown SNP detection sequence
ttgaaaacttcttctcacaaatactatatgagggcacactacacagttggt

SEQ ID NO: 8 - DNA, 51 bp, Unknown
  Description of Unknown SNP detection sequence
ttgaaaacttcttctcacaaatactctatgagggcacactacacagttggt

SEQ ID NO: 9 - DNA, 51 bp, Unknown
  Description of Unknown SNP detection sequence
ttggtcataaatgactgacacgtgaatccatacacaatttggagatgggtt

SEQ ID NO: 10 - DNA, 51 bp, Unknown
  Description of Unknown SNP detection sequence
ttggtcataaatgactgacacgtgactccatacacaatttggagatgggtt